(19) United States
(12) Patent Application Publication
(10) Pub. No.: US 2025/0059317 A1
(43) Pub. Date: Feb. 20, 2025

(54) POLYMERIC CYCLOALIPHATIC EPOXIDES

(71) Applicant: Arkema France, Colombes (FR)

(72) Inventors: Kelly SQUIRES, Wetherby (GB); Petr SEHNAL, Wetherby (GB); Sen LIU, Wetherby (GB); Richard PLENDERLEITH, Wetherby (GB); Kangtai REN, Exton, PA (US)

(21) Appl. No.: 18/723,943

(22) PCT Filed: Dec. 23, 2022
(86) PCT No.: PCT/EP2022/087808

§ 371 Date: Jun. 25, 2024

(30) Foreign Application Priority Data
Dec. 30, 2021 (FR) 2114670

(51) Int. Cl. C08G 59/32; C09D 163/00; C09J 163/00

(52) U.S. Cl. CPC C08G 59/3218; C09D 163/00; C09J 163/00

(57) ABSTRACT

The present invention relates to an alkoxylated cycloaliphatic epoxide according to the following formula (I) wherein each R₁ and R₂ is independently selected from H and Me; L is the residue of a polyol; each a is independently from 2 to 4; each b is independently 0 to 20 with the proviso that at least one b is not 0; c is at least 3. The invention also relates to compositions comprising a compound of formula (I) and a process for the preparation of a cured product. Cured products according to the invention are in particular 3D-printed articles.

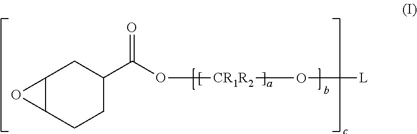

### FIELD OF THE INVENTION

The present invention relates to an alkoxylated cycloaliphatic epoxide and a process for its preparation, compositions containing such an alkoxylated cycloaliphatic epoxide, processes for curing such compositions, cured products thus obtained and uses of such products, notably as 3D-printed articles.

### BACKGROUND OF THE INVENTION

In the field of photocure 3D printing, polymerizable products are required showing reduced toxicity, such as mutagenic risks, as well as high performance in terms of features such as the speed of curing, resistance to surface degradation by frictional contact of cured resins (rubbing) etc.

The following difunctional cycloaliphatic epoxide, sold as UviCure® S105 gives clear, hard, glossy coatings:

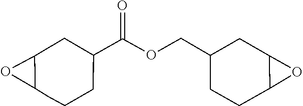 [SMILES: O=C(OCC1CCC2OC2C1)C1CCCC2OC2C1]

Although this epoxide usually gives good cure speed, brittle mechanical properties may be observed.

Generally speaking, the viscosity profile of a resin may require effective management. For example, 3D printing applications may require a longer “sit time”, such that initially thin epoxy resins would run and spread too much. Resistance to solvent is also a feature to be optimized with respect to existing resins, as well as curing efficiency, for example curing speed maintained at lower lamp power for UV-curable compositions, with respect to existing epoxy resins.

### SUMMARY OF THE INVENTION

A first aspect of the invention is an alkoxylated cycloaliphatic epoxide according to the following formula (I):

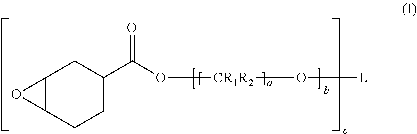

wherein
each R₁ and R₂ is independently selected from H and Me;
L is the residue of a polyol;
each a is independently from 2 to 4;
each b is independently 0 to 20 with the proviso that at least one b is not 0;
c is at least 3.

Another aspect of the invention is a process for the preparation of an alkoxylated cycloaliphatic epoxide of formula (I) as defined above, wherein the process comprises the following steps:
a) reacting a cyclohexene of formula (VI) with an alkoxylated polyol of formula (VII) to obtain an alkoxylated cyclohexene of formula (VIII);
b) epoxidation of the alkoxylated cyclohexene of formula (VIII) to obtain an alkoxylated cycloaliphatic epoxide of formula (I);

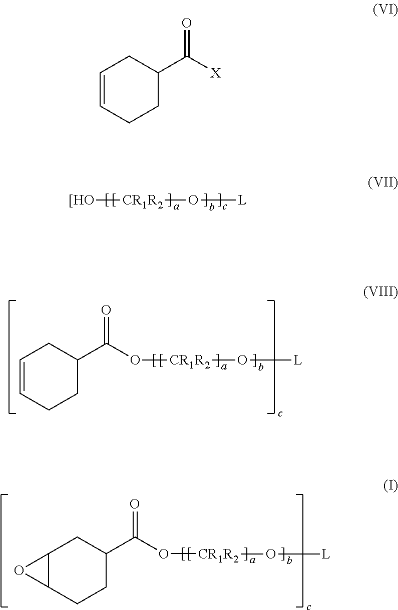

wherein
L, R₁, R₂, a, b and c are as defined above;
X is OH, O-Alk or;
Alk is a C1-C6 alkyl.

Further aspects of the invention concern a composition comprising at least one alkoxylated cycloaliphatic epoxide according to the formula (I) set out above.

Yet another aspect of the invention concerns a process for the preparation of a cured product, comprising curing such a composition, in particular by exposing the composition to radiation such as UV, near-UV, visible, infrared and/or near-infrared radiation or to an electron beam.

Yet another aspect of the invention concerns a cured product obtained by curing a composition according to the invention. The cured product may be used as an ink, a coating, a sealant, an adhesive, a molded article or a 3D-printed article, in particular a 3D-printed article.

### DETAILED DESCRIPTION

### Description of Figures

FIG. 1 shows the results of tensile stress measurements on cured resin materials according to the present invention, and on cured resin materials not according to the present invention.

FIG. 2 shows the results of storage modulus measurements on cured resin materials according to the present invention, and on cured resin materials not according to the present invention.

FIG. 3 shows the results of heat flow measurements on cured resin materials according to the present invention, and on cured resin materials not according to the present invention.

### DEFINITIONS

In the present application, the term “comprise(s) a/an” means “comprise(s) one or more”. Unless mentioned otherwise, the % by weight in a compound or a composition are expressed based on the weight of the compound, respectively of the composition.

The term «alkyl» means a monovalent saturated hydrocarbon radical of formula —CₙH₂ₙ₊₁. An alkyl may be linear or branched. A «C1-C20 alkyl» means an alkyl having 1 to 20 carbon atoms. Examples of alkyl groups include methyl, ethyl, propyl, isopropyl, butyl, isobutyl, tert-butyl, pentyl and hexyl.

The term «alkylaryl» means an alkyl substituted by an aryl group. A «C7-C20 alkylaryl» means an alkylaryl having 7 to 20 carbon atoms. An example of an alkylaryl group is benzyl (—CH₂-Phenyl).

The term «halogen» means an atom selected from Cl, Br and I.

The term «alkylene» means a divalent saturated hydrocarbon radical of formula —CₙH₂ₙ—. An alkylene may be linear or branched. A «C1-C20 alkylene» means an alkylene having 1 to 20 carbon atoms. Examples of alkylene groups include ethylene (—CH₂—CH₂—) and 1,2-propylene (—CH₂—CH(CH₃)—).

The term «alkenyl» means a monovalent unsaturated hydrocarbon radical. An alkenyl may be linear or branched. A «C2-C20 alkenyl» means an alkenyl having 2 to 20 carbon atoms. Examples of alkenyl groups include vinyl (—CH═CH₂) and allyl (—CH₂—CH═CH₂).

The term «cycloalkyl» means a monovalent saturated alicyclic hydrocarbon radical comprising a cycle. A «C3-C8 cycloalkyl» means a cycloalkyl having 3 to 8 carbon atoms. Examples of cycloalkyl groups include cyclopentyl, cyclohexyl and isobornyl.

The term «alkoxy» means a group of formula —O-Alkyl.

The term «aryl» means an aromatic hydrocarbon group. A «C6-C12 aryl» means an aryl having 6 to 12 carbon atoms.

The term «heteroaryl» means an aromatic group comprising a heteroatom such as O, N, S and mixtures thereof. A «C5-C9 heteroaryl» means a heteroaryl having 5 to 9 carbon atoms.

The term «polyol» means a compound comprising at least two hydroxyl groups.

The term «polyester» means a compound comprising at least two ester bonds.

The term «polyether» means a compound comprising at least two ether bonds.

The term «polycarbonate» means a compound comprising at least two carbonate bonds.

The term «polyester polyol» means a polyester comprising at least two hydroxyl groups.

The term «polyether polyol» means a polyether comprising at least two hydroxyl groups.

The term «polycarbonate polyol» means a polycarbonate comprising at least two hydroxyl groups.

The term «hydrocarbon radical» means a radical consisting of carbon and hydrogen atoms.

Unless mentioned otherwise a hydrocarbon radical is not substituted or interrupted by any heteroatoms (O, N or S). A hydrocarbon radical may be linear or branched, saturated or unsaturated, aliphatic, cycloaliphatic or aromatic.

The term «hydroxyl group» means a —OH group.

The term «amine» means a —NRₐR_{b} group, wherein Rₐ and R_{b} are independently H or a C1-C6 alkyl. The term «primary amine» means a —NH₂ group. The term «secondary amine» means a —NHRₐ group wherein Rₐ is a C1-C6 alkyl. The term «tertiary amine» means a —NRₐR_{b} group, wherein Rₐ and R_{b} are independently a C1-C6 alkyl.

The term «carboxylic acid» means a-COOH group.

The term «isocyanate group» means a —N═C═O group.

The term «ester bond» means a —C(═O)—O— or —O—C(═O)— bond.

The term «ether bond» means a —O— bond.

The term «carbonate bond» means a —O—C(═O)—O-bond.

The term «urethane or carbamate» means a —NH—C(═O)—O— or —O—C(═O)—NH-bond.

The term «amide bond» means a —C(═O)—NH— or —NH—C(═O)— bond.

The term «urea bond» means a —NH—C(═O)—NH-bond.

The term «polyisocyanate» means a compound comprising at least two isocyanate groups.

The term «aliphatic» means a non-aromatic acyclic compound. It may be linear or branched, saturated or unsaturated. It may be substituted by one or more groups, for example selected from alkyl, hydroxyl, halogen (Br, Cl, I), isocyanate, carbonyl, amine, carboxylic acid, —C(═O)—OR′, —C(═O)—O—C(═O)—R′, each R′ being independently a C1-C6 alkyl. It may comprise one or more bonds selected from ether, ester, amide, urethane, urea and combinations thereof.

The term «acyclic» means a compound that does not comprise any rings

The term «cycloaliphatic» means a non-aromatic cyclic compound. It may be substituted by one or more groups as defined for the term «aliphatic». It may comprise one or more bonds as defined for the term «aliphatic».

The term «aromatic» means a compound comprising an aromatic ring, which means that it respects Hückel's aromaticity rule, in particular a compound comprising a phenyl group. It may be substituted by one or more groups as defined for the term «aliphatic». It may comprise one or more bonds as defined for the term «aliphatic».

The term «saturated» means a compound that does not comprise any double or triple carbon-carbon bonds.

The term «unsaturated» means a compound that comprises a double or triple carbon-carbon bond, in particular a double carbon-carbon bond.

The term «optionally substituted» means a compound substituted by one or more groups selected from alkyl, cycloalkyl, aryl, heteroaryl, alkoxy, alkylaryl, haloalkyl, hydroxyl, halogen, isocyanate, nitrile, amine, amide, carboxylic acid, —C(═O)—R′—C(═O)—OR′, —C(═O)NH—R′, —NH—C(═O)R′, —O—C(═O)—NH—R′, —NH—C(═O)—O—R′, —C(═O)—O—C(═O)—R′ and —SO₂—NH—R′, each R′ being independently an optionally substituted group selected from alkyl, aryl and alkylaryl. The term «3D article» means a three-dimensional object obtained by 3D printing.

Compounds of the Invention and Processes for their Synthesis

Epoxy resins can be polymerized, for example by cationic polymerization and, as mentioned above, cycloaliphatic epoxides such as those based on a cyclohexane ring are known. In the present invention, a polyol core is attached to multiple epoxide-bearing C6 cycloaliphatic groups through ethyleneoxy (—CH₂—CH₂—O—), 1,2-propyleneoxy (—CH₂—CH(CH₃)—O—) or analogous spacers.

The invention thus relates in one aspect to an alkoxylated cycloaliphatic epoxide according to the following formula (I):

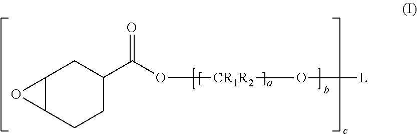

wherein
each R₁ and R₂ is independently selected from H and Me;
L is the residue of a polyol;
each a is independently from 2 to 4;
each b is independently 0 to 20 with the proviso that at least one b is not 0;
c is at least 3.

In particular, the value for c may be 3, 4, 5, 6, 7, 8, 9 or 10. In one embodiment, c may be equal to 3. In another embodiment c may be higher than 3, for example c may be from 4 to 10. Before curing, the alkoxylated cycloaliphatic epoxide compounds of the present invention thus contain multiple epoxide groups separated from one another and available for curing.

In an illustrative example of an alkoxylated cycloaliphatic epoxide of the present invention, which is a preferred example, but to which the present invention is not limited, the alkoxylated cycloaliphatic epoxide may have the following structure:

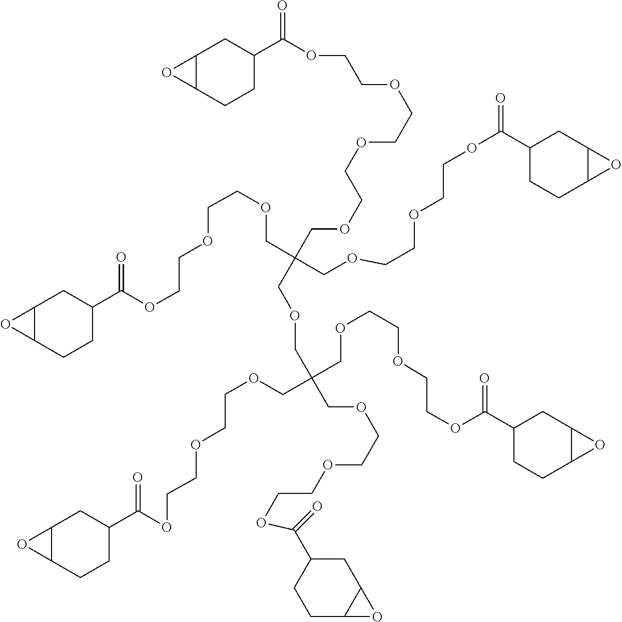 [SMILES: O=C(OCCOCCOCCOCC(COCCOCCOC(=O)C1CCC2OC2C1)(COCCOCCOC(=O)C1CCC2OC2C1)COCC(COCCOCCOC(=O)C1CCC2OC2C1)(COCCOCCOC(=O)C1CCC2OC2C1)COCCOCCOC(=O)C1CCC2OC2C1)C1CCC2OC2C1]

The above preferred hexafunctional molecule is derived from (HO—CH₂—)₃C—CH₂)₂O. The latter polyol is commercially available from Perstorp AB as Polyol R6405, its systematic name is poly(oxy-1,2-ethanediyl), α-hydro-ω-hydroxy-, ether with 2,2′-[oxybis(methylene)]bis[2-(hydroxymethyl)-1,3-propanediol](6:1). Its CAS # is 50977-32-7.

Generally speaking, among preferred alkoxylated cycloaliphatic epoxides of the invention, falling within general formula (I) above, are ones wherein a is 2, the alkoxylated cycloaliphatic epoxide being according to the following formula (Ia):

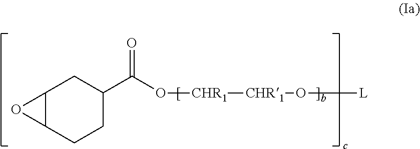

wherein
L, b and c are as defined in claim 1;
each R₁ and R′₁ is independently selected from H and Me.

Another type of preferred alkoxylated cycloaliphatic epoxide of the invention, falling within general formula (I) above, shows a as 4 and R₁ and R₂ are both H.

In preferred alkoxylated cycloaliphatic epoxides of the invention, according to the different preferences set out above for a, and for R₁ and R′₁, or R₁ and R₂, each b is independently from 1 to 20, in particular from 1 to 10, more particularly from 2 to 6. In preferred alkoxylated cycloaliphatic epoxides of the invention, the alkoxylated cycloaliphatic epoxide has an alkoxylation degree of at least 6, in particular at least 8, more particularly at least 10, even more particularly at least 12.

In preferred alkoxylated cycloaliphatic epoxides of the invention, c is from 3 to 10, in particular from 3 to 8, more particularly from 4 to 6.

In preferred alkoxylated cycloaliphatic epoxides of the invention, c is from 4 to 10, in particular from 4 to 8, more particularly from 4 to 6, even more particularly c may be equal to 6. Alternatively, c may be equal to 3.

In preferred alkoxylated cycloaliphatic epoxides of the invention, c is 3 and L is a trivalent linker according to the following formula (II):

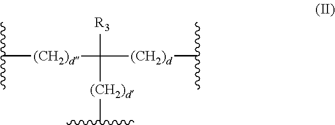

wherein
R₃ is selected from H, alkyl and alkoxy, in particular R₃ is alkyl, more particularly R₃ is ethyl;
d, d′ and d″ are independently 0 to 2 with the proviso that at least 2 among d, d′ and d″ are not 0, in particular d, d′ and d″ are all 1 or d is 0 and d′ and d″ are 1.

In other preferred alkoxylated cycloaliphatic epoxides of the invention, c is 4 and L is a tetravalent linker according to one of the following formulae (IIIa), (IIIb) or (IIIc):

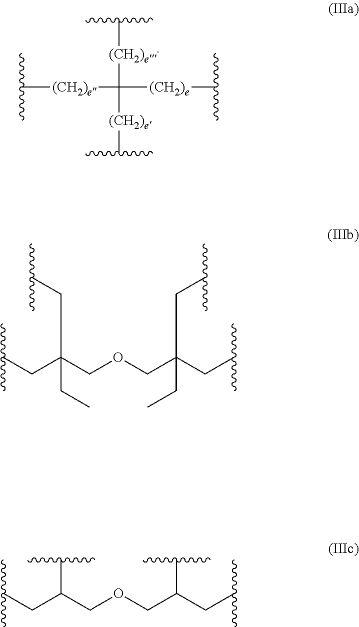

wherein
e, e′, e″ and e″′ are independently 0 to 2 with the proviso that at least 3 among e, e′, e″ and e″′ are not 0, in particular e, e′, e″ and e″′ are all 1.

In other preferred alkoxylated cycloaliphatic epoxides of the invention, c is 5 and L is a pentavalent linker according to the following formula (IV):

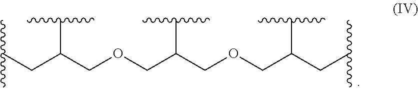 [SMILES: *CC(*)COCC(*)COCC(*)C*]

In other preferred alkoxylated cycloaliphatic epoxides of the invention, c is 6 and L is a hexavalent linker according to the following formula (Va), (Vb) or (Vc):

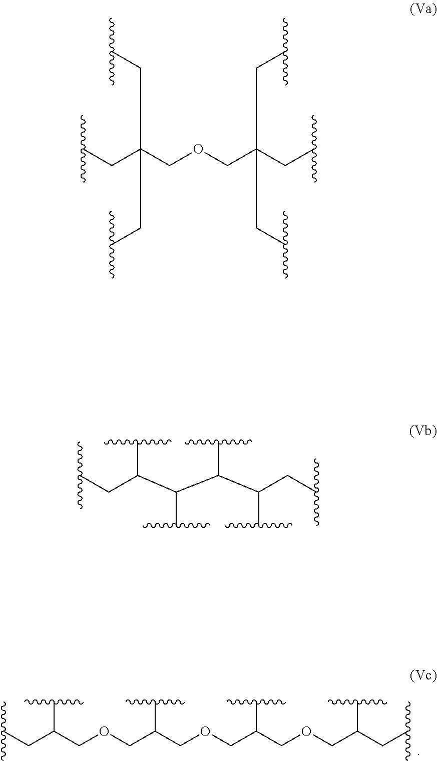 [SMILES: *CC(C*)(C*)COCC(C*)(C*)C* ; *CC(*)C(*)C(*)C(*)C* ; *CC(*)COCC(*)COCC(*)COCC(*)C*]

In processes according to the present invention, esterification is carried out, forming an ester of an alkoxylated polyol and a cyclohexene bearing a carboxylic acid, followed by subsequent reaction with an epoxidation agent in an epoxidation step, converting the multiple cyclohexene C═C groups into epoxide groups.

Thus, in a process for the preparation of an alkoxylated cycloaliphatic epoxide of formula (I) of the present invention as defined above, the process comprises the following steps:
a) reacting a cyclohexene of formula (VI) with an alkoxylated polyol of formula (VII) to obtain an alkoxylated cyclohexene of formula (VIII);
b) epoxidation of the alkoxylated cyclohexene of formula (VIII) to obtain an alkoxylated cycloaliphatic epoxide of formula (I) as defined above

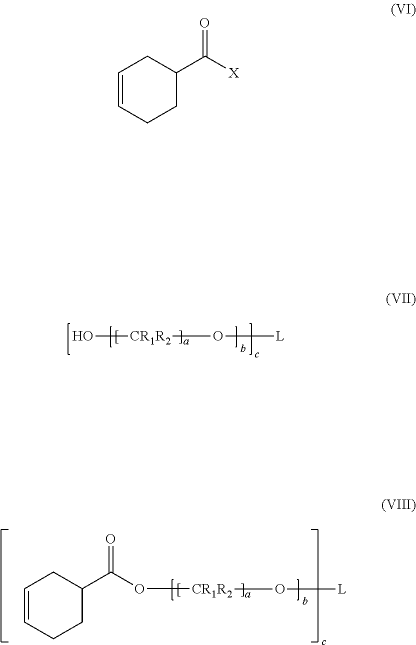

wherein
L, R₁, R₂, a, b and c are as defined above in relation to the alkoxylated cycloaliphatic epoxide compounds of the present invention;
X is OH, O-Alk or Cl;
Alk is a C1-C6 alkyl.

As set out above, the alkoxylated polyol of formula (VII) may be linked to form the ester (VIII) either by direct esterification with 3-cyclohexene-1-carboxylic acid, or via an intermediate such as the acid chloride cyclohex-3-ene-1-carbonyl chloride. The latter acid chloride may be obtained by reaction of 3-cyclohexene-1-carboxylic acid with an agent such as thionyl chloride, phosphorus trichloride, phosphorus(V) oxychloride and oxalyl chloride.

Epoxidation step (b) may be carried out with a peracid such as 3-chloroperbenzoic acid, peracetic acid or with other epoxidation agents such as hydrogen peroxide, t-butyl hydroperoxide and sodium hypochlorite.

### Compositions of the Invention

Compositions of the present invention include, but are not limited to, compositions comprising:
a) at least one alkoxylated cycloaliphatic epoxide of formula (I) as defined above; and
b) at least one cationically polymerizable compound other than component a).

Component b) may notably be selected from the group consisting of an oxetane, an oxolane, a cyclic acetal, a cyclic lactone, a thiirane, a thietane, a spiro orthoester, a spiro orthocarbonate, a vinyl ether, a vinyl ester, derivatives thereof and mixtures thereof. Oxetanes are particularly preferred examples of component (b). Components (b), such as oxetanes, may serve as reactive diluents and provide high curing speed and also high solvent resistance in compositions with (a) at least one alkoxylated cycloaliphatic epoxide of formula (I).

The weight ratio between component a) and component b) may be from 20:80 to 80:20, in particular from 30:70 to 70:30, more particularly from 40:60 to 60:40.

In this first type of preferred composition in the present invention, comprising a) at least one alkoxylated cycloaliphatic epoxide of formula (I) as defined above; and b) at least one cationically polymerizable compound, such as an oxetane, the composition preferably comprises at least one cationic photoinitiator, in particular an onium salt or a metallocene salt, more particularly a halonium salt, a sulfonium salt (e.g. a triarylsulfonium salt such as triarylsulfonium hexafluoroantimonate salt), a sulfoxonium salt, a diazonium salt, a ferrocene salt, and mixtures thereof.

### Cationically Polymerizable Compound

As mentioned above, in a first preferred option, the composition of the invention may further comprise, in addition to at least one alkoxylated cycloaliphatic epoxide of formula (I), a cationically polymerizable compound b), such as an oxetane, and/or c) polyols. The composition of the invention may comprise a mixture of cationically polymerizable compounds b) and c).

When the composition comprises a cationically polymerizable compound, the composition may be a hybrid free-radical/cationic composition, i.e. a composition that is cured by free radical polymerization and cationic polymerization.

The term “cationically polymerizable compound” means a compound comprising a polymerizing functional group which polymerizes via a cationic mechanism, for example a heterocyclic group or a carbon-carbon double bond substituted with an electrodonating group. In a cationic polymerization mechanism, a cationic initiator forms a Brønsted or Lewis acid species that binds to the cationically polymerizable compound which then becomes reactive and leads to chain growth by reaction with another cationically polymerizable compound.

The cationically polymerizable compound may be selected from epoxy-functionalized compounds, oxetanes, oxolanes, cyclic acetals, cyclic lactones, thiiranes, thietanes, spiro orthoesters, ethylenically unsaturated compounds other than (meth)acrylates, derivatives thereof and mixtures thereof.

In a preferred embodiment, the cationically polymerizable compound may be selected from epoxy-functionalized compounds, oxetanes and mixtures thereof. In particular, oxetanes are preferred cationically polymerizable compounds in compositions of the present invention.

Suitable epoxy-functionalized compounds capable of being cationically polymerized are glycidyl ethers, in particular mono-, di-, tri- and polyglycidyl ether compounds, and alicyclic epoxy compounds including those comprising residue of carboxylic acids such as, for example, alkylcarboxylic acid residual groups, alkylcycloalkylcarboxylic acid residual groups and alkylene dicarboxylic acid residual groups. For example, the epoxy-functionalized compounds may be bisphenol A diglycidyl ether, bisphenol F diglycidyl ether, bisphenol S diglycidyl ether, brominated bisphenol A diglycidyl ether, brominated bisphenol F diglycidyl ether, brominated bisphenol S diglycidyl ether, epoxy novolak resin, hydrogenated bisphenol A diglycidyl ether, hydrogenated bisphenol F diglycidyl ether, hydrogenated bisphenol S diglycidyl ether, 3,4-epoxycyclohexylmethyl-3′,4′-epoxycyclohexanecarboxylate, 2-(7-oxabicyclo[4.1.0]heptan-3-yl)spiro[1,3-dioxane-5,3′-7-oxabicyclo[4.1.0]heptane], bis(3,4-epoxycyclohexylmethyl)adipate, vinylcyclohexene oxide, 4-vinylepoxycyclohexane, 4-vinylcyclohexene dioxide, bis(3,4-epoxy-6-methylcyclohexylmethyl)adipate, 3,4-epoxy-6-methylcyclohexyl-3′,4′-epoxy-6′-methylcyclohexanecarboxylate, methylenebis(3,4-epoxycyclohexane), dicyclopentadiene diepoxide, di(3,4-epoxycyclohexylmethyl) ether of ethylene glycol, ethylenebis(3,4-epoxycyclohexanecarboxylate), epoxyhexahydrodioctylphthalate, epoxyhexahydro-di-2-ethylhexyl phthalate, 1,4-butanediol diglycidyl ether, 1,6-hexanediol diglycidyl ether, glycerol triglycidyl ether, trimethylolpropane triglycidyl ether, polyethylene glycol diglycidyl ether, polypropylene glycol diglycidyl ether, polyglycidyl ethers of polyether polyol obtained by the addition of one or more alkylene oxides to aliphatic polyhydric alcohols such as ethylene glycol, propylene glycol, and glycerol, diglycidyl esters of aliphatic long-chain dibasic acids, monoglycidyl ethers of aliphatic higher alcohols, monoglycidyl ethers of phenol, cresol, butyl phenol, or polyether alcohols obtained by the addition of alkylene oxide to these compounds, glycidyl esters of higher fatty acids, epoxidized soybean oil, epoxybutylstearic acid, epoxyoctylstearic acid, epoxidized linseed oil, epoxidized polybutadiene, and the like.

Suitable oxetanes capable of being cationically polymerized include trimethylene oxide, 3,3-dimethyloxetane, 3,3-dichloromethyloxetane, 3-ethyl-3-phenoxymethyloxetane, and bis(3-ethyl-3-methyloxy)butane, 3-ethyl-3-oxetanemethanol.

Suitable oxolanes capable of being cationically polymerized include tetrahydrofuran and 2,3-dimethyltetrahydrofuran.

Suitable cyclic acetals capable of being cationically polymerized include trioxane, 1,3-dioxolane, and 1,3,6-trioxacyclooctane.

Suitable cyclic lactones capable of being cationically polymerized include f-propiolactone and ε-caprolactone.

Suitable thiiranes capable of being cationically polymerized include ethylene sulfide, 1,2-propylene sulfide, and thioepichlorohydrin.

Suitable thietanes capable of being cationically polymerized include 3,3-dimethylthietane.

Suitable spiro orthoesters capable of being cationically polymerized are compounds obtained by the reaction of an epoxy compound and a lactone.

Suitable ethylenically unsaturated compounds other than (meth)acrylates capable of being cationically polymerized include vinyl ethers such as ethylene glycol divinyl ether, triethylene glycol divinyl ether and trimethylolpropane trivinyl ether.

In a preferred embodiment, component b) comprises at least one oxetane, in particular at least one oxetane according to the following formula (IX):

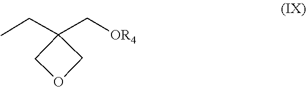

wherein R₄ is selected from H, alkyl, aryl, alkylaryl, (meth)acryloyl, —CH₂-oxetanyl-CH₂—CH₃, L₁-O—CH₂-oxetanyl-CH₂—CH₃;
L₁ is a divalent linker, in particular-CH₂-Ph-Ph-CH₂ or —CH₂-Ph-CH₂—[O—CH₂-Ph-CH₂]_{f}—
Ph is phenylene;
f is0 to 10.

In particular, component b) may comprise at least one oxetane according to formula (IX) wherein R₄ is H, benzyl or —CH₂-oxetanyl-CH₁CH₃;
preferably R₄ is H or —CH₂-oxetanyl-CH₂—CH₃.

The curable composition of the invention may comprise 10 to 80%, in particular 15 to 75%, more particularly 20 to 70%, by weight of cationically polymerizable compound based on the total weight of the curable composition.

### Hybrid Free-Radical/Cationic Compositions

In a further preferred composition option in the present invention, the composition may be one that is cured by both free radical polymerization and cationic polymerization. In preferred embodiments with (a) alkoxylated cycloaliphatic epoxide according to above cited formula (I), and optionally further an oxetane (b) on the one-hand, and a monomer able to take part in C═C addition polymerization on the other hand, for example a (meth)acrylate group-containing compound, normally, interpenetrating (intertwined) networks of separate polyepoxide and poly(meth)acrylate are obtained with no covalent bonds between them. However, it may be advantageous to have a composition containing a monomer component containing both epoxide/oxetane and (meth)acrylate groups. In that case covalent links are formed between the two networks which may provide further improvement of physical properties. Examples or such commercially available compounds include UViCure S170 (3-Ethyl-3-(Methacryloyloxy)Methyloxetane) and glycidyl (meth)acrylate.

Thus, in a second type of preferred composition of the invention, the composition comprises: a) at least one alkoxylated cycloaliphatic epoxide of formula (I) as set out above or a composition comprising at least one alkoxylated cycloaliphatic epoxide of formula (I) and b) at least one cationically polymerizable compound other than component a), notably an oxetane, an oxolane, a cyclic acetal, a cyclic lactone, a thiirane, a thietane, a spiro orthoester, a spiro orthocarbonate, a vinyl ether, a vinyl ester, derivatives thereof and mixtures thereof; and c) at least one (meth)acrylate-functionalized compound, in particular a (meth)acrylate-functionalized compound bearing at least 2 or at least 3 (meth)acrylate groups.

In this second type of preferred composition of the invention, cationically curable oxetanes are optional components, although they may be used in preferred embodiments. Other optional components are cationically curable curable vinylethers, polyols or multifunctional alcohols, which may function as chain transfer agents.

As used herein, the term “(meth)acrylate-functionalized compound” means a monomer comprising a (meth)acrylate group, in particular an acrylate group. The term “(meth)acrylate-functionalized compound” here encompasses containing more than one (meth)acrylate group, such as 2, 3, 4, 5 or 6 (meth)acrylate groups, commonly referred to as “oligomers” comprising a (meth)acrylate group. The term “(meth)acrylate group” encompasses acrylate groups (—O—CO—CH═CH₂) and methacrylate groups (—O—CO—C(CH₃)═CH₂).

Preferably, the (meth)acrylate-functionalized compound does not comprise any amino group. As used herein, the term “amino group” refers to a primary, secondary or tertiary amine group, but does not include any other type of nitrogen-containing group such as an amide, carbamate (urethane), urea, or sulfonamide group).

The (meth)acrylate-functionalized compound may have a molecular weight of less than 600 g/mol, in particular from 100 to 550 g/mol, more particularly 200 to 500 g/mol.

The (meth)acrylate-functionalized compound may have 1 to 6 (meth)acrylate groups, in particular 1 to 5 (meth)acrylate groups, more particularly 1 to 3 (meth)acrylate groups.

The (meth)acrylate-functionalized compounds may comprise a mixture of (meth)acrylate-functionalized monomers having different functionalities. For example the (meth)acrylate-functionalized compound may comprise a mixture of a (meth)acrylate-functionalized compound containing a single acrylate or methacrylate group per molecule (referred to herein as “mono(meth)acrylate-functionalized compounds”) and a (meth)acrylate-functionalized compound containing 2 or more, preferably 2 or 3, acrylate and/or methacrylate groups per molecule. In another example, the (meth)acrylate-functionalized compounds may comprise a mixture of at least one mono(meth)acrylate-functionalized compound and at least one (meth)acrylate-functionalized compound containing 3 or more, preferably 4 or more, (meth)acrylate groups per molecule.

The (meth)acrylate functionalized compound may comprise a mono(meth)acrylate-functionalized compound. The mono(meth)acrylate-functionalized compound may advantageously function as a reactive diluent and reduce the viscosity of the composition of the invention.

Examples of suitable mono(meth)acrylate-functionalized compounds include, but are not limited to, mono-(meth)acrylate esters of aliphatic alcohols (wherein the aliphatic alcohol may be straight chain, branched or alicyclic and may be a mono-alcohol, a di-alcohol or a polyalcohol, provided only one hydroxyl group is esterified with (meth)acrylic acid); mono-(meth)acrylate esters of aromatic alcohols (such as phenols, including alkylated phenols); mono-(meth)acrylate esters of alkylaryl alcohols (such as benzyl alcohol); mono-(meth)acrylate esters of oligomeric and polymeric glycols such as diethylene glycol, triethylene glycol, dipropylene glycol, tripropylene glycol, polyethylene glycol, and polypropylene glycol); mono-(meth)acrylate esters of monoalkyl ethers of glycols and oligoglycols; mono-(meth)acrylate esters of alkoxylated (e.g., ethoxylated and/or propoxylated) aliphatic alcohols (wherein the aliphatic alcohol may be straight chain, branched or alicyclic and may be a mono-alcohol, a di-alcohol or a polyalcohol, provided only one hydroxyl group of the alkoxylated aliphatic alcohol is esterified with (meth)acrylic acid); mono-(meth)acrylate esters of alkoxylated (e.g., ethoxylated and/or propoxylated) aromatic alcohols (such as alkoxylated phenols); caprolactone mono(meth)acrylates; and the like.

The following compounds are specific examples of mono(meth)acrylate-functionalized compounds suitable for use in the curable compositions of the present invention: methyl (meth)acrylate; ethyl (meth)acrylate; n-propyl (meth)acrylate; n-butyl (meth)acrylate; isobutyl (meth)acrylate; n-hexyl (meth)acrylate; 2-ethylhexyl (meth)acrylate; n-octyl (meth)acrylate; isooctyl (meth)acrylate; n-decyl (meth)acrylate; n-dodecyl (meth)acrylate; tridecyl (meth)acrylate; tetradecyl (meth)acrylate; hexadecyl (meth)acrylate; 2-hydroxyethyl (meth)acrylate; 2- and 3-hydroxypropyl (meth)acrylate; 2-methoxyethyl (meth)acrylate; 2-ethoxyethyl (meth)acrylate; 2- and 3-ethoxypropyl (meth)acrylate; tetrahydrofurfuryl (meth)acrylate; alkoxylated tetrahydrofurfuryl (meth)acrylate; 2-(2-ethoxyethoxy)ethyl (meth)acrylate; cyclohexyl (meth)acrylate; glycidyl (meth)acrylate; isodecyl (meth)acrylate; lauryl (meth)acrylate; 2-phenoxyethyl (meth)acrylate; alkoxylated phenol (meth)acrylates; alkoxylated nonylphenol (meth)acrylates; cyclic trimethylolpropane formal (meth)acrylate; isobornyl (meth)acrylate; tricyclodecanemethanol (meth)acrylate; tert-butylcyclohexanol (meth)acrylate; trimethylcyclohexanol (meth)acrylate; diethylene glycol monomethyl ether (meth)acrylate; diethylene glycol monoethyl ether (meth)acrylate; diethylene glycol monobutyl ether (meth)acrylate; triethylene glycol monoethyl ether (meth)acrylate; ethoxylated lauryl (meth)acrylate; methoxy polyethylene glycol (meth)acrylates; 3-(2-hydroxyalkyl)oxazolidinone (meth)acrylates; and combinations thereof. The (meth)acrylate functionalized compound may comprise a (meth)acrylate-functionalized compound containing two or more (meth)acrylate groups per molecule.

Examples of suitable (meth)acrylate-functionalized compound containing two or more (meth)acrylate groups per molecule include acrylate and methacrylate esters of polyhydric alcohols (organic compounds containing two or more, e.g., 2 to 6, hydroxyl groups per molecule). Specific examples of suitable polyhydric alcohols include C₂₋₂₀ alkylene glycols (glycols having a C₂₋₁₀ alkylene group may be preferred, in which the carbon chain may be branched; e.g., ethylene glycol, trimethylene glycol, 1,2-propylene glycol, 1,2-butanediol, 1,3-butanediol, 2,3-butanediol, tetramethylene glycol (1,4-butanediol), 1,5-pentanediol, 1,6-hexanediol, 1,8-octanediol, 1,9-nonanediol, 1,12-dodecanediol, cyclohexane-1,4-dimethanol, bisphenols, and hydrogenated bisphenols, as well as alkoxylated (e.g., ethoxylated and/or propoxylated) derivatives thereof), diethylene glycol, glycerin, alkoxylated glycerin, triethylene glycol, dipropylene glycol, tripropylene glycol, trimethylolpropane, alkoxylated trimethylolpropane, ditrimethylolpropane, alkoxylated ditrimethylolpropane, pentaerythritol, alkoxylated pentaerythritol, dipentaerythritol, alkoxylated dipentaerythritol, cyclohexanediol, alkoxylated cyclohexanediol, cyclohexanedimethanol, alkoxylated cyclohexanedimethanol, norbornene dimethanol, alkoxylated norbornene dimethanol, norbornane dimethanol, alkoxylated norbornane dimethanol, polyols containing an aromatic ring, cyclohexane-1,4-dimethanol ethylene oxide adducts, bis-phenol ethylene oxide adducts, hydrogenated bisphenol ethylene oxide adducts, bisphenol propylene oxide adducts, hydrogenated bisphenol propylene oxide adducts, cyclohexane-1,4-dimethanol propylene oxide adducts, sugar alcohols and alkoxylated sugar alcohols. Such polyhydric alcohols may be fully or partially esterified (with (meth)acrylic acid, (meth)acrylic anhydride, (meth)acryloyl chloride or the like), provided they contain at least two (meth)acrylate functional groups per molecule. As used herein, the term “alkoxylated” refers to compounds containing one or more oxyalkylene moieties (e.g., oxyethylene and/or oxypropylene moieties). An oxyalkylene moiety corresponds to the general structure —R—O—, wherein R is a divalent aliphatic moiety such as —CH₂CH₂— or —CH₂CH(CH₃)—. For example, an alkoxylated compound may contain from 1 to 30 oxyalkylene moieties per molecule.

Exemplary (meth)acrylate-functionalized compounds containing two or more (meth)acrylate groups per molecule may include bisphenol A di(meth)acrylate; hydrogenated bisphenol A di(meth)acrylate; ethylene glycol di(meth)acrylate; diethylene glycol di(meth)acrylate; triethylene glycol di(meth)acrylate; tetraethylene glycol di(meth)acrylate; polyethylene glycol di(meth)acrylate; propylene glycol di(meth)acrylate; dipropylene glycol di(meth)acrylate; tripropylene glycol di(meth)acrylate; tetrapropylene glycol di(meth)acrylate; polypropylene glycol di(meth)acrylate; polytetramethylene glycol di(meth)acrylate; 1,2-butanediol di(meth)acrylate; 2,3-butanediol di(meth)acrylate; 1,3-butanediol di(meth)acrylate; 1,4-butanediol di(meth)acrylate; 1,5-pentanediol di(meth)acrylate; 1,6-hexanediol di(meth)acrylate; 1,8-octanediol di(meth)acrylate; 1,9-nonanediol di(meth)acrylate; 1,10-nonanediol di(meth)acrylate; 1,12-dodecanediol di(meth)acrylate; neopentyl glycol di(meth)acrylate; 2-methyl-2,4-pentanediol di(meth)acrylate; polybutadiene di(meth)acrylate; cyclohexane-1,4-dimethanol di(meth)acrylate; tricyclodecane dimethanol di(meth)acrylate; metallic di(meth)acrylates; modified metallic di(meth)acrylates; glyceryl di(meth)acrylate; glyceryl tri(meth)acrylate; trimethylolethane tri(meth)acrylate; trimethylolethane di(meth)acrylate; trimethylolpropane tri(meth)acrylate; trimethylolpropane di(meth)acrylate; pentaerythritol di(meth)acrylate; pentaerythritol tri(meth)acrylate; pentaerythritol tetra(meth)acrylate, di(trimethylolpropane) diacrylate; di(trimethylolpropane) triacrylate; di(trimethylolpropane) tetraacrylate, sorbitol penta(meth)acrylate; di(pentaerythritol) tetraacrylate; di(pentaerythritol) pentaacrylate; di(pentaerythritol) hexa(meth)acrylate; tris (2-hydroxyethyl) isocyanurate tri(meth)acrylate; as well as the alkoxylated (e.g., ethoxylated and/or propoxylated) derivatives thereof; and combinations thereof.

The curable composition of the invention may comprise 10 to 80%, in particular 15 to 75%, more particularly 20 to 70%, by weight of (meth)acrylate-functionalized compound based on the total weight of the curable composition.

The (meth)acrylate-functionalized compound in the form of an oligomer may be selected in order to enhance the flexibility, strength and/or modulus, among other attributes, of a cured polymer prepared using the curable composition of the present invention. Here, the (meth)acrylate functionalized oligomer may have up to 18 (meth)acrylate groups, in particular 2 to 6 (meth)acrylate groups, more particularly 2 to 6 acrylate groups. The (meth)acrylate functionalized compound in the form of an oligomer may have a number average molecular weight equal or more than 600 g/mol, in particular 800 to 15,000 g/mol, more particularly 1,000 to 5,000 g/mol.

In particular, the (meth)acrylate-functionalized compounds in the form of an oligomer may be selected from the group consisting of (meth)acrylate-functionalized epoxy oligomers (sometimes also referred to as “epoxy (meth)acrylate oligomers”), (meth)acylate-functionalized polyether oligomers (sometimes also referred to as “polyether (meth)acrylate oligomers”), (meth)acrylate-functionalized polydiene oligomers (sometimes also referred to as “polydiene (meth)acrylate oligomers”), (meth)acrylate-functionalized polycarbonate oligomers (sometimes also referred to as “polycarbonate (meth)acrylate oligomers”), and (meth)acrylate-functionalized polyester oligomers (sometimes also referred to as “polyester (meth)acrylate oligomers”) and mixtures thereof.

Exemplary polyester (meth)acrylate oligomers include the reaction products of acrylic or methacrylic acid or mixtures or synthetic equivalents thereof with hydroxyl group-terminated polyester polyols. The reaction process may be conducted such that all or essentially all of the hydroxyl groups of the polyester polyol have been (meth)acrylated, particularly in cases where the polyester polyol is difunctional. The polyester polyols can be made by polycondensation reactions of polyhydroxyl functional components (in particular, diols) and polycarboxylic acid functional compounds (in particular, dicarboxylic acids and anhydrides). The polyhydroxyl functional and polycarboxylic acid functional components can each have linear, branched, cycloaliphatic or aromatic structures and can be used individually or as mixtures.

Examples of suitable epoxy (meth)acrylates include the reaction products of acrylic or methacrylic acid or mixtures thereof with an epoxy resin (polyglycidyl ether or ester). The epoxy resin may, in particular, by selected from bisphenol A diglycidyl ether, bisphenol F diglycidyl ether, bisphenol S diglycidyl ether, brominated bisphenol A diglycidyl ether, brominated bisphenol F diglycidyl ether, brominated bisphenol S diglycidyl ether, epoxy novolak resin, hydrogenated bisphenol A diglycidyl ether, hydrogenated bisphenol F diglycidyl ether, hydrogenated bisphenol S diglycidyl ether, 3,4-epoxycyclohexylmethyl-3′,4′-epoxycyclohexanecarboxylate, 2-(7-oxabicyclo[4.1.0]heptan-3-yl)spiro[1,3-dioxane-5,3′-7-oxabicyclo[4.1.0]heptane], bis(3,4-epoxycyclohexylmethyl)adipate, vinylcyclohexene oxide, 4-vinylepoxycyclohexane, bis(3,4-epoxy-6-methylcyclohexylmethyl)adipate,3,4-epoxy-6-methylcyclohexyl-3′,4′-epoxy-6′-methylcyclohexanecarboxylate, methylenebis(3,4-epoxycyclohexane), dicyclopentadiene diepoxide, di(3,4-epoxycyclohexylmethyl) ether of ethylene glycol, ethylenebis(3,4-epoxycyclohexanecarboxylate), 1,4-butanediol diglycidyl ether, 1,6-hexanediol diglycidyl ether, glycerol triglycidyl ether, trimethylolpropane triglycidyl ether, polyethylene glycol diglycidyl ether, polypropylene glycol diglycidyl ether, polyglycidyl ethers of a polyether polyol obtained by the addition of one or more alkylene oxides to an aliphatic polyhydric alcohol such as ethylene glycol, propylene glycol, and glycerol, diglycidyl esters of aliphatic long-chain dibasic acids, monoglycidyl ethers of aliphatic higher alcohols, monoglycidyl ethers of phenol, cresol, butyl phenol, or polyether alcohols obtained by the addition of alkylene oxide to these compounds, glycidyl esters of higher fatty acids, epoxidized soybean oil, epoxybutylstearic acid, epoxyoctylstearic acid, epoxidized linseed oil, epoxidized polybutadiene, and the like.

Suitable polyether (meth)acrylate oligomers include, but are not limited to, the condensation reaction products of acrylic or methacrylic acid or synthetic equivalents or mixtures thereof with polyetherols which are polyether polyols (such as polyethylene glycol, polypropylene glycol or polytetramethylene glycol). Suitable polyetherols can be linear or branched substances containing ether bonds and terminal hydroxyl groups. Polyetherols can be prepared by ring opening polymerization of cyclic ethers such as tetrahydrofuran or alkylene oxides (e.g., ethylene oxide and/or propylene oxide) with a starter molecule. Suitable starter molecules include water, polyhydroxyl functional materials, and polyester polyols.

Suitable acrylic (meth)acrylate oligomers (sometimes also referred to in the art as “acrylic oligomers”) include oligomers which may be described as substances having an oligomeric acrylic backbone which is functionalized with one or (meth)acrylate groups (which may be at a terminus of the oligomer or pendant to the acrylic backbone). The acrylic backbone may be a homopolymer, random copolymer or block copolymer comprised of repeating units of acrylic monomers. The acrylic monomers may be any monomeric (meth)acrylate such as C1-C6 alkyl (meth)acrylates as well as functionalized (meth)acrylates such as (meth)acrylates bearing hydroxyl, carboxylic acid and/or epoxy groups. Acrylic (meth)acrylate oligomers may be prepared using any procedures known in the art, such as by oligomerizing monomers, at least a portion of which are functionalized with hydroxyl, carboxylic acid and/or epoxy groups (e.g., hydroxyalkyl(meth)acrylates, (meth)acrylic acid, glycidyl (meth)acrylate) to obtain a functionalized oligomer intermediate, which is then reacted with one or more (meth)acrylate-containing reactants to introduce the desired (meth)acrylate functional groups.

The curable composition of the invention may comprise 10 to 80%, in particular 15 to 75%, more particularly 20 to 70%, by weight of (meth)acrylate-functionalized compound based on the total weight of the curable composition.

Non-limiting types of radical photoinitiators suitable for use in the curable compositions of the present invention include, for example, benzoins, benzoin ethers, acetophenones, o-hydroxy acetophenones, benzyl, benzyl ketals, anthraquinones, phosphine oxides, acylphosphine oxides, α-hydroxyketones, phenylglyoxylates, α-aminoketones, benzophenones, thioxanthones, xanthones, acridine derivatives, phenazene derivatives, quinoxaline derivatives, triazine compounds, benzoyl formates, aromatic oximes, metallocenes, acylsilyl or acylgermanyl compounds, camphorquinones, polymeric derivatives thereof, and mixtures thereof.

Examples of suitable radical photoinitiators include, but are not limited to, 2-methylanthraquinone, 2-ethylanthraquinone, 2-chloroanthraquinone, 2-benzyanthraquinone, 2-t-butylanthraquinone, 1,2-benzo-9,10-anthraquinone, benzyl, benzoins, benzoin ethers, benzoin, benzoin methyl ether, benzoin ethyl ether, benzoin isopropyl ether, alpha-methylbenzoin, alpha-phenylbenzoin, Michler's ketone, acetophenones such as 2,2-dialkoxybenzophenones and 1-hydroxyphenyl ketones, benzophenone, 4,4′-bis-(diethylamino) benzophenone, acetophenone, 2,2-diethyloxyacetophenone, diethyloxyacetophenone, 2-isopropyithioxanthone,thioxanthone, diethyl thioxanthone, 1,5-acetonaphthylene, benzil ketone, α-hydroxy keto, 2,4,6-trimethylbenzoyldiphenyl phosphine oxide, benzyl dimethyl ketal, 2,2-dimethoxy-1,2-diphenylethanone, 1-hydroxycylcohexyl phenyl ketone, 2-methyl-1-[4-(methylthio) phenyl]-2-morpholinopropanone-1,2-hydroxy-2-methyl-1-phenyl-propanone, oligomeric α-hydroxy ketone, benzoyl phosphine oxides, phenylbis(2,4,6-trimethylbenzoyl)phosphine oxide, ethyl(2,4,6-trimethylbenzoyl)phenyl phosphinate, anisoin, anthraquinone, anthraquinone-2-sulfonic acid, sodium salt monohydrate, (benzene) tricarbonylchromium, benzil, benzoin isobutyl ether, benzophenone/1-hydroxycyclohexyl phenyl ketone, 50/50 blend, 3,3′,4,4′-benzophenonetetracarboxylic dianhydride, 4-benzoylbiphenyl, 2-benzyl-2-(dimethylamino)-4′-morpholinobutyrophenone, 4,4′-bis(diethylamino)benzophenone, 4,4′-bis(dimethylamino)benzophenone, camphorquinone, 2-chlorothioxanthen-9-one, dibenzosuberenone, 4,4′-dihydroxybenzophenone, 2,2-dimethoxy-2-phenylacetophenone, 4-(dimethylamino)benzophenone, 4,4′-dimethylbenzil, 2,5-dimethylbenzophenone, 3,4-dimethylbenzophenone, diphenyl(2,4,6-trimethylbenzoyl)phosphine oxide/2-hydroxy-2-methylpropiophenone, 50/50 blend, 4-ethoxyacetophenone, 2,4,6-trimethylbenzoyldiphenylphophine oxide, phenyl bis(2,4,6-trimethyl benzoyl)phosphine oxide, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, (cumene)cyclopentadienyl iron(ii) hexafluorophosphate, 9,10-diethoxy and 9,10-dibutoxyanthracene, 2-ethyl-9,10-dimethoxyanthracene, thioxanthen-9-one and combinations thereof.

In preferred compositions of the invention, a radical photoinitiator having Norrish type I activity may be used, such as a phosphine oxide. Acetophenone family photoinitiators are also a preferred choice in hybrid systems containing both cationically polymerizable compounds including alkoxylated cycloaliphatic epoxide of formula (I) and radical polymerizable (meth)acrylate-functionalized compounds.

The amount of photoinitiator(s) may be from 0.01% to 5%, from 0.02% to 3%, from 0.05 to 2%, from 0.1 to 1.5% or from 0.2 to 1%, by weight based on the total weight of the curable composition. The total amount of photoinitiator(s) may be from 0.01 to 10%, from 0.1 to 9%, from 0.2 to 8%, from 0.5 to 7% or from 1 to 6%, by weight based on the total weight of the curable composition.

### Additives

The curable composition of the present invention may comprise an additive. The curable composition may comprise a mixture of additives.

In particular, the additive may be selected from sensitizers, amine synergists, antioxidants/photostabilizers, light blockers/absorbers, polymerization inhibitors, foam inhibitors, flow or leveling agents, colorants, pigments, dispersants (wetting agents, surfactants), slip additives, fillers, chain transfer agents, thixotropic agents, matting agents, impact modifiers, waxes, mixtures thereof, and any other additives conventionally used in the coating, sealant, adhesive, molding, 3D printing or ink arts.

The curable composition may comprise a sensitizer.

Sensitizers may be introduced in the curable composition of the present invention in order to extend the sensitivity of the photoinitiator to longer wavelengths. For example, the sensitizer may absorb light at longer or shorter wavelengths than the photoinitiator and be capable of transferring the energy to the photoinitiator and revert to its ground state.

Examples of suitable sensitizers include anthracenes and carbazoles.

The concentration of sensitizer in the curable composition will vary depending on the photoinitiator that is used. Typically, however, the curable composition is formulated to comprise from 0% to 5%, in particular 0.1% to 3%, more particularly 0.5 to 2%, by weight of sensitizer based on the total weight of the curable composition.

The curable composition may comprise a chain-transfer agent.

Chain-transfer agents may be introduced in the curable composition of the present invention in order to increase the curing speed. In particular, the chain-transfer agent may be a polyol. Polythiols or polyamines may slow down cationic cure and are not a preferred choice in the present invention.

The curable composition may comprise a stabilizer.

Stabilizers may be introduced in the curable composition of the present invention in order to provide adequate storage stability and shelf life. Advantageously, one or more such stabilizers are present at each stage of the method used to prepare the curable composition, to protect against unwanted reactions during processing of the ethylenically unsaturated components of the curable composition. As used herein, the term “stabilizer” means a compound or substance which retards or prevents reaction or curing of actinically-curable functional groups present in a composition in the absence of actinic radiation. However, it will be advantageous to select an amount and type of stabilizer such that the composition remains capable of being cured when exposed to actinic radiation (that is, the stabilizer does not prevent radiation curing of the composition). Typically, effective stabilizers for purposes of the present invention will be classified as free radical stabilizers (i.e., stabilizers which function by inhibiting free radical reactions).

Any of the stabilizers known in the art related to (meth)acrylate-functionalized compounds may be utilized in the present invention. Quinones represent a particularly preferred type of stabilizer which can be employed in the context of the present invention. As used herein, the term “quinone” includes both quinones and hydroquinones as well as ethers thereof such as monoalkyl, monoaryl, monoaralkyl and bis(hydroxyalkyl) ethers of hydroquinones. Hydroquinone monomethyl ether is an example of a suitable stabilizer which can be utilized. Other stabilizers known in the art such as BHT and derivatives, phosphite compounds, phenothiazine (PTZ), triphenyl antimony and tin(II) salts can also be used.

The concentration of stabilizer in the curable composition will vary depending upon the particular stabilizer or combination of stabilizers selected for use and also on the degree of stabilization desired and the susceptibility of components in the curable compositions towards degradation in the absence of stabilizer. Typically, however, the curable composition is formulated to comprise from 5 to 5000 ppm stabilizer. According to certain embodiments of the invention, the reaction mixture during each stage of the method employed to make the curable composition contains at least some stabilizer, e.g., at least 10 ppm stabilizer.

The curable composition may comprise a light blocker (sometimes referred to as a light absorber). The introduction of a light blocker is particularly advantageous when the curable composition is to be used as a resin in a three-dimensional printing process involving photocuring of the curable composition. The light blocker may be any such substances known in the three-dimensional printing art, including for example non-reactive pigments and dyes. The light blocker may be a visible light blocker or a UV light blocker, for example. Examples of suitable light blockers include, but are not limited to, titanium dioxide, carbon black and organic ultraviolet light absorbers such as hydroxybenzophenone, hydroxyphenylbenzotriazole, oxanilide, hydroxyphenyltriazine, Sudan I, bromothymol blue, 2,2′-(2,5-thiophenediyl)bis(5-tert-butylbenzoxazole) (sold under the brand name “Benetex OB Plus”) and benzotriazole ultraviolet light absorbers.

The amount of light blocker may be varied as may be desired or appropriate for particular applications. Generally speaking, if the curable composition contains a light blocker, it is present in a concentration of from 0.001 to 10% by weight based on the weight of the curable composition.

Advantageously, the curable compositions of the present invention may be formulated to be solvent-free, i.e., free of any non-reactive volatile substances (substances having a boiling point at atmospheric pressure of 150° C. or less). For example, the curable compositions of the present invention may contain little or no non-reactive solvent, e.g., less than 10% or less than 5% or less than 1% or even 0% non-reactive solvent, based on the total weight of the curable composition. As used herein, the term non-reactive solvent means a solvent that does not react when exposed to the actinic radiation used to cure the curable compositions described herein.

According to other advantageous embodiments of the invention, the curable composition is formulated to be useable as a one component or one part system. That is, the curable composition is cured directly and is not combined with another component or second part (such as an amine monomer, as defined in U.S. Pat. Application Publication No. 2017/0260418 A1) prior to being cured.

### Curable Composition

In preferred embodiments of the invention, the curable composition is a liquid at 25° C. In various embodiments of the invention, the curable compositions described herein are formulated to have a viscosity of less than 10,000 mPa·s (cP), or less than 5,000 mPa·s (cP), or less than 4,000 mPa·s (cP), or less than 3,000 mPa·s (cP), or less than 2,500 mPa·s (cP), or less than 2,000 mPa·s (cP), or less than 1,500 mPa·s (cP), or less than 1,000 mPa·s (cP) or even less than 500 mPa·s (cP) as measured at 25° C. using a Brookfield viscometer, model DV-II, using a 27 spindle (with the spindle speed varying typically between 20 and 200 rpm, depending on viscosity). In advantageous embodiments of the invention, the viscosity of the curable composition is from 200 to 5,000 mPa·s (cP), or from 200 to 2,000 mPa·s (cP), or from 200 to 1,500 mPa·s (cP), or from 200 to 1,000 mPa·s (cP) at 25° C. Relatively high viscosities can provide satisfactory performance in applications where the curable composition is heated above 25° C., such as in three-dimensional printing operations or the like which employ machines having heated resin vats.

The curable compositions described herein may be compositions that are to be subjected to curing by means of free radical polymerization, cationic polymerization or other types of polymerization. In particular embodiments, the curable compositions are photocured (i.e., cured by exposure to actinic radiation such as light, in particular visible or UV light).

The curable composition of the invention may be an ink, coating, sealant, adhesive, molding, or 3D printing composition, in particular a 3D-printing composition.

End use applications for the curable compositions include, but are not limited to, inks, coatings, adhesives, additive manufacturing resins (such as 3D printing resins), molding resins, sealants, composites, antistatic layers, electronic applications, recyclable materials, smart materials capable of detecting and responding to stimuli, packaging materials, personal care articles, articles for use in agriculture, water or food processing, or animal husbandry, and biomedical materials. The curable compositions of the invention thus find utility in the production of biocompatible articles.

Such articles may, for example, exhibit high biocompatibility, low cytotoxicity and/or low extractables.

The composition according to the invention may in particular be used to obtain a cured product, a 3D printed article according to the following processes.

### Process for the Preparation of a Cured Product, a 3D-Printed Article

The process for the preparation of a cured product according to the invention comprises curing the composition of the invention. In particular, the composition may be cured by exposing the composition to radiation. More particularly, the composition may be cured by exposing the composition to UV, near-UV, visible, infrared and/or near-infrared radiation or to an electron beam. Curing may be accelerated or facilitated by supplying energy to the curable composition, such as by heating the curable composition. Thus, the cured product may be deemed the reaction product of the curable composition, formed by curing. A curable composition may be partially cured by exposure to actinic radiation, with further curing being achieved by heating the partially cured article. For example, an article formed from the curable composition (e.g., a 3D printed article) may be heated at a temperature of from 40° C. to 120° C. for a period of time of from 5 minutes to 12 hours.

Prior to curing, the curable composition may be applied to a substrate surface in any known conventional manner, for example, by spraying, knife coating, roller coating, casting, drum coating, dipping, and the like and combinations thereof. Indirect application using a transfer process may also be used. A substrate may be any commercially relevant substrate, such as a high surface energy substrate or a low surface energy substrate, such as a metal substrate or plastic substrate, respectively. The substrates may comprise metal, paper, cardboard, glass, thermoplastics such as polyolefins, polycarbonate, acrylonitrile butadiene styrene (ABS), and blends thereof, composites, wood, leather and combinations thereof. When used as an adhesive, the curable composition may be placed between two substrates and then cured, the cured composition thereby bonding the substrates together to provide an adhered article. Curable compositions in accordance with the present invention may also be formed or cured in a bulk manner (e.g., the curable composition may be cast into a suitable mold and then cured).

The cured product obtained with the process of the invention may be an ink, a coating, a sealant, an adhesive, a molded article or a 3D-printed article.

In particular, the cured product may be a 3D-printed article. A 3D-printed article may be defined as an article obtained with a 3D-printer using a computer-aided design (CAD) model or a digital 3D model.

The 3D-printed article may, in particular, be obtained with a process for the preparation of a 3D-printed article that comprises printing a 3D article with the composition of the invention. In particular, the process may comprise printing a 3D article layer by layer or continuously.

A plurality of layers of a curable composition in accordance with the present invention may be applied to a substrate surface; the plurality of layers may be simultaneously cured (by exposure to a single dose of radiation, for example) or each layer may be successively cured before application of an additional layer of the curable composition.

The curable compositions which are described herein can be used as resins in three-dimensional printing applications. Three-dimensional (3D) printing (also referred to as additive manufacturing) is a process in which a 3D digital model is manufactured by the accretion of construction material. The 3D printed object is created by utilizing the computer-aided design (CAD) data of an object through sequential construction of two dimensional (2D) layers or slices that correspond to cross-sections of 3D objects. Stereolithography (SL) is one type of additive manufacturing where a liquid resin is hardened by selective exposure to a radiation to form each 2D layer. The radiation can be in the form of electromagnetic waves or an electron beam. The most commonly applied energy source is UV, near-UN, visible, infrared and/or near-infrared radiation.

Non-limiting examples of suitable 3D printing processes include stereolithography (SLA); digital light process (DLP); liquid crystal device (LCD); inkjet head (or multijet) printing; Continuous Liquid Interface Production (CLIP); extrusion type processes such as continuous fiber 3D printing and cast-in-motion 3D printing; and volumetric 3D printing. The building method may be “layer by layer” or continuous. The liquid may be in a vat, or deposited with an inkjet or gel deposition, for example.

Stereolithography and other photocurable 3D printing methods typically apply low intensity light sources to radiate each layer of a photocurable resin to form the desired article. As a result, photocurable resin polymerization kinetics and the green strength of the printed article are important criteria if a particular photocurable resin will sufficiently polymerize (cure) when irradiated and have sufficient green strength to retain its integrity through the 3D printing process and post-processing.

The curable compositions of the invention are especially useful as 3D printing resin formulations, that is, compositions intended for use in manufacturing three-dimensional articles using 3D printing techniques. Such three-dimensional articles may be free-standing/self-supporting and may consist essentially of or consist of a composition in accordance with the present invention that has been cured. The three-dimensional article may also be a composite, comprising at least one component consisting essentially of or consisting of a cured composition as previously mentioned as well as at least one additional component comprised of one or more materials other than such a cured composition (for example, a metal component or a thermoplastic component or inorganic filler or fibrous reinforcement). The curable compositions of the present invention are particularly useful in digital light printing (DLP), although other types of three-dimensional (3D) printing methods may also be practiced using the inventive curable compositions (e.g., SLA, inkjet, multi-jet printing, piezoelectric printing, actinically-cured extrusion, and gel deposition printing). The curable compositions of the present invention may be used in a three-dimensional printing operation together with another material which functions as a scaffold or support for the article formed from the curable composition of the present invention.

Thus, the curable compositions of the present invention are useful in the practice of various types of three-dimensional fabrication or printing techniques, including methods in which construction of a three-dimensional object is performed in a step-wise or layer-by-layer manner. In such methods, layer formation may be performed by solidification (curing) of the curable composition under the action of exposure to radiation, such as visible, UV or other actinic irradiation. For example, new layers may be formed at the top surface of the growing object or at the bottom surface of the growing object. The curable compositions of the present invention may also be advantageously employed in methods for the production of three-dimensional objects by additive manufacturing wherein the method is carried out continuously. For example, the object may be produced from a liquid interface. Suitable methods of this type are sometimes referred to in the art as “continuous liquid interface (or interphase) product (or printing)” (“CLIP”) methods. Such methods are described, for example, in WO 2014/126830; WO 2014/126834; WO 2014/126837; and Tumbleston et al., “Continuous Liquid Interface Production of 3D Objects,” Science Vol. 347, Issue 6228, pp. 1349-1352 (Mar. 20, 2015), the entire disclosure of which is incorporated herein by reference in its entirety for all purposes.

When stereolithography is conducted above an oxygen-permeable build window, the production of an article using a curable composition in accordance with the present invention may be enabled in a CLIP procedure by creating an oxygen-containing “dead zone” which is a thin uncured layer of the curable composition between the window and the surface of the cured article as it is being produced. In such a process, a curable composition is used in which curing (polymerization) is inhibited by the presence of molecular oxygen; such inhibition is typically observed, for example, in curable compositions which are capable of being cured by free radical mechanisms. The dead zone thickness which is desired may be maintained by selecting various control parameters such as photon flux and the optical and curing properties of the curable composition. The CLIP process proceeds by projecting a continuous sequence of actinic radiation (e.g., UV) images (which may be generated by a digital light-processing imaging unit, for example) through an oxygen-permeable, actinic radiation-(e.g., UV-) transparent window below a bath of the curable composition maintained in liquid form. A liquid interface below the advancing (growing) article is maintained by the dead zone created above the window. The curing article is continuously drawn out of the curable composition bath above the dead zone, which may be replenished by feeding into the bath additional quantities of the curable composition to compensate for the amounts of curable composition being cured and incorporated into the growing article.

In another embodiment, the curable composition will be supplied by ejecting it from a printhead rather than supplying it from a vat. This type of process is commonly referred to as inkjet or multijet 3D printing. One or more UV curing sources mounted just behind the inkjet printhead cures the curable composition immediately after it is applied to the build surface substrate or to previously applied layers. Two or more printheads can be used in the process which allows application of different compositions to different areas of each layer. For example, compositions of different colors or different physical properties can be simultaneously applied to create 3D printed parts of varying composition. In a common usage, support materials-which are later removed during post-processing—are deposited at the same time as the compositions used to create the desired 3D printed part. The printheads can operate at temperatures from about 25° C. up to about 100° C. Viscosities of the curable compositions are less than 30 mPa·s at the operating temperature of the printhead.

The process for the preparation of a 3D-printed article may comprise the steps of:
a) providing (e.g., coating) a first layer of a curable composition in accordance with the present invention onto a surface;
b) curing the first layer, at least partially, to provide a cured first layer;
c) providing (e.g., coating) a second layer of the curable composition onto the cured first layer;
d) curing the second layer, at least partially, to provide a cured second layer adhered to the cured first layer; and
e) repeating steps c) and d) a desired number of times to build up the three-dimensional article.

Although the curing steps may be carried out by any suitable means, which will in some cases be dependent upon the components present in the curable composition, in certain embodiments of the invention the curing is accomplished by exposing the layer to be cured to an effective amount of radiation, in particular actinic radiation (e.g., electron beam radiation, UV radiation, visible light, etc.). The three-dimensional article which is formed may be heated in order to effect thermal curing.

Accordingly, in various embodiments, the present invention provides a process comprising the steps of:
a) providing (e.g., coating) a first layer of a curable composition in accordance with the present invention and in liquid form onto a surface;
b) exposing the first layer imagewise to actinic radiation to form a first exposed imaged cross-section, wherein the radiation is of sufficient intensity and duration to cause at least partial curing of the layer in the exposed areas;
c) providing (e.g., coating) an additional layer of the curable composition onto the previously exposed imaged cross-section;
d) exposing the additional layer imagewise to actinic radiation to form an additional imaged cross-section, wherein the radiation is of sufficient intensity and duration to cause at least partial curing of the additional layer in the exposed areas and to cause adhesion of the additional layer to the previously exposed imaged cross-section;
e) repeating steps c) and d) a desired number of times to build up the three-dimensional article.

Alternatively, the process for the preparation of a 3D-printed article may comprise the steps of:
a) providing a carrier and an optically transparent member having a build surface, the carrier and build surface defining a build region therebetween;
b) filling the build region with a composition as defined above;
c) continuously or intermittently curing part of the composition in the build region according to the method as defined above to form a cured composition; and
d) continuously or intermittently advancing the carrier away from the build surface to form the 3D-printed article from the cured composition.

After the 3D article has been printed, it may be subjected to one or more post-processing steps. The post-processing steps can be selected from one or more of the following steps removal of any printed support structures, washing with water and/or organic solvents to remove residual resins, and post-curing using thermal treatment and/or actinic radiation either simultaneously or sequentially. The post-processing steps may be used to transform the freshly printed article into a finished, functional article ready to be used in its intended application.

The cured product and 3D-printed articles obtained with the processes of the invention are described herein after.

### Cured Product, 3D-Printed Article

The cured product of the invention is obtained by curing the composition of the invention or according to the process of the invention.

The cured product may be an ink, a coating, a sealant, an adhesive, a molded article or a 3D-printed article. In particular, the cured product may be a 3D-printed article.

### Uses

The alkoxylated cycloaliphatic epoxide of the invention may be used to obtain an ink, a coating, a sealant, an adhesive, a molded article or a 3D-printed article, in particular a 3D-printed article. Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

In some embodiments, the invention herein can be construed as excluding any element or process step that does not materially affect the basic and novel characteristics of the invention. Additionally, in some embodiments, the invention can be construed as excluding any element or process step not specified herein.

Although the invention is illustrated and described herein with reference to specific embodiments, the invention is not intended to be limited to the details shown. Rather, various modifications may be made in the details within the scope and range of equivalents of the claims and without departing from the invention.

The invention is illustrated with the following non-limiting examples.

### EXAMPLES

### Materials

All materials used in the examples are readily available from standard commercial sources such as Sigma-Aldrich Company Ltd. and Tokyo Chemical Industry Ltd. unless otherwise specified. Polyol R3215, Polyol 4360 and Polyol R6405 are available from Perstorp AB.

### Example 1: Synthesis of a Triepoxide Derived from Polyol R3215 (E1)

### Synthesis of Cyclohex-3-ene-1-carbonyl chloride

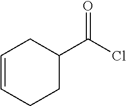

3-cyclohexene-1-carboxylic acid (400.0 g, 3.1708 mol) is dissolved in chloroform (1870 mL) under nitrogen and N,N-dimethylformamide (10 mL) is added.

To the stirred reaction mixture is added thionyl chloride (456.0 g; 3.8329 mol) over 7 h. HCl and SO₂ gases are evolved, the internal temperature is maintained at 15-22° C. during the addition. After the addition is complete, the reaction mixture is stirred for 18 h under nitrogen at 20° C. Chloroform, DMF and unreacted thionyl chloride are then removed in vacuo. The pale yellow liquid product is then dried to constant weight in vacuo (30 mbar, 30° C.). Any precipitated white solid is removed by filtration. This provides cyclohex-3-ene-1-carbonyl chloride (451.5 g; 98.5% of theory).

¹H NMR (400 MHz, CDCl₃): 5.70 (m, 2H), 3.04-2.97 (m, 1H), 2.46-2.29 (m, 2H), 2.22-2.07 (m, 3H), 1.87-1.76 (m, 1H).

### Synthesis of a trialkene derived from Polyol R3215

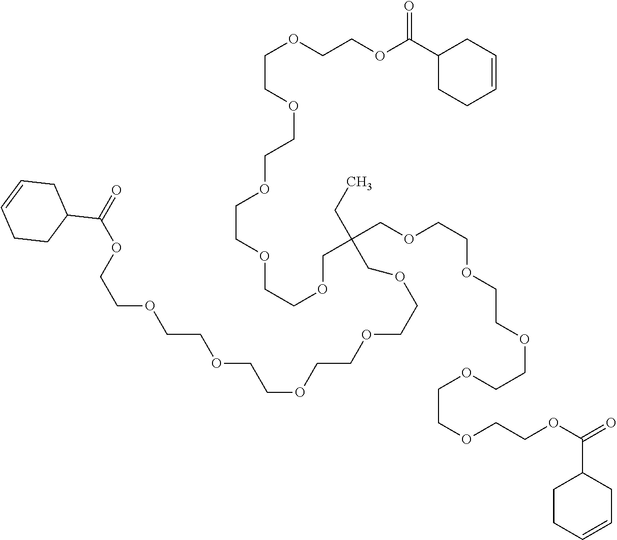 [SMILES: CCC(COCCOCCOCCOCCOCCOC(=O)C1CC=CCC1)(COCCOCCOCCOCCOCCOC(=O)C1CC=CCC1)COCCOCCOCCOCCOCCOC(=O)C1CC=CCC1]

A reaction vessel is charged with Polyol R3215 (100.0 g; 125.8 mmol) and the material is dried under vacuum with stirring (20 mbar, 75° C., 3 h). The reaction vessel is then cooled to 20° C., flushed with nitrogen and charged with dichloromethane (500 mL) and triethylamine (50.91 g, 503.1 mmol).

The clear solution is then cooled with ice-water to 5° C. and cyclohex-3-ene-1-carbonyl chloride (78.4 g, 542 mmol) is added over 40 min while maintaining internal temperature at 5° C. (the reaction is exothermic). The turbid reaction mixture is then allowed to warm to 20° C. over 2 h and stirred at this temperature for a further 18 h.

The reaction mixture is then charged into a solution of sodium hydrogen carbonate (100 g) in water (1000 mL) and stirred rapidly for 6 h at 20° C. The organic phase is separated and extracted with water (2×350 mL). The organic phase is collected and all volatiles are removed in vacuo. The liquid product is then dried to constant weight in vacuo (30 mbar, 50° C.). This provides the desired trialkene product (147.6 g).

¹H NMR (400 MHz, CDCl₃): 5.68 (m, 6H), 4.25 (m, 6H), 3.71-3.53 (m, 54H), 3.32 (m, 6H), 2.63-2.54 (m, 3H), 2.39-1.95 (m, 15H), 1.75-1.63 (m, 3H), 1.45-1.36 (m, 2H), 0.87-0.81 (m, 3H).

### Synthesis of a Triepoxide Derived from Polyol 3215(E1)

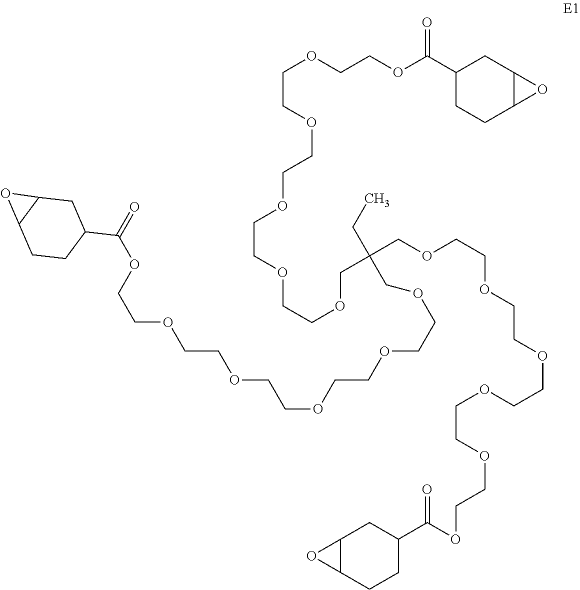 [SMILES: CCC(COCCOCCOCCOCCOCCOC(=O)C1CCC2OC2C1)(COCCOCCOCCOCCOCCOC(=O)C1CCC2OC2C1)COCCOCCOCCOCCOCCOC(=O)C1CCC2OC2C1]

A reaction vessel is charged with the Polyol R3215 trialkene (147.6 g, 131.9 mmol) and dichloromethane (1100 mL) is added. The reaction mixture is cooled to an internal temperature of 3° C. 3-chloroperbenzoic acid (73.5% active, 99.1 g, 422.1 mmol) is added over 5 h with vigorous stirring, while maintaining the internal temperature at 3-4° C. The turbid mixture is stirred vigorously for 18 h and allowed to warm to 20° C.

The reaction mass is then filtered and the white solid is washed with dichloromethane (2×50 mL). To the obtained filtrate is added a solution of sodium sulphite (50 g) in water (500 mL) and the biphasic mixture is stirred for 60 min. The mixture is phase separated and the organic phase is washed with a solution of sodium hydrogen carbonate (83.6 g) in water (750 mL), and then with water (2×500 mL).

The mixture is allowed to phase separate for 18 h and the organic phase is collected, dried with sodium sulphate (100 g) and filtered. The filtrate is concentrated in vacuo and the liquid product is then dried to constant weight (30 mbar, 35° C.).

This provides the desired triepoxide product E1 (141.5 g, 91.9% of theory).

¹H NMR (400 MHz, CDCl₃): 4.22 (m, 6H), 3.80-3.54 (m, 54H), 3.31 (m, 6H), 3.25-3.14 (m, 6H), 2.58-2.50 (m, 3H), 2.32-1.88 (m, 12H), 1.82-1.74 (m, 3H), 1.68-1.56 (m, 2H), 1.48-1.35 (m, 3H), 0.86-0.81 (m, 3H).

FT-IR (ATR, neat): 2866 (m), 1729 (s), 1303 (m), 1254 (m), 1232 (m), 1173 (m), 1099 (vs),1061 (m), 991 (m), 973 (m), 938 (m), 873 (m), 796 (m).

### Example 2: Synthesis of a Tetraepoxide Derived from Polyol 4360 (E21

### Synthesis of a Tetraalkene Derived from Polyol 4360

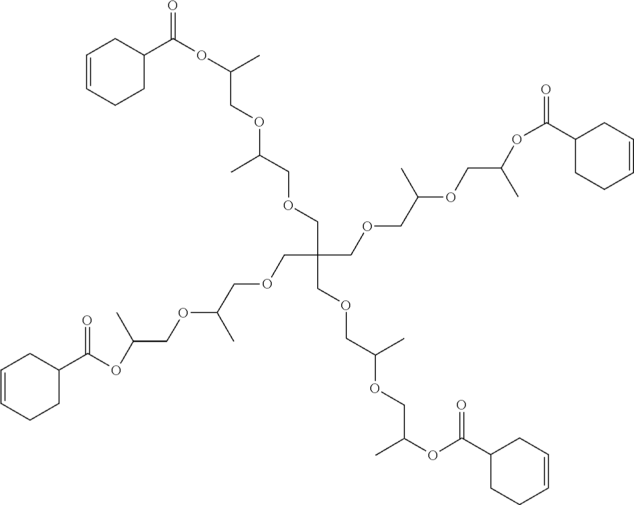 [SMILES: CC(COCC(COCC(C)OCC(C)OC(=O)C1CC=CCC1)(COCC(C)OCC(C)OC(=O)C1CC=CCC1)COCC(C)OCC(C)OC(=O)C1CC=CCC1)OCC(C)OC(=O)C1CC=CCC1]

A reaction vessel is charged with Polyol 4360 (99.0 g, 157.1 mmol) and the material is dried under vacuum with stirring (20 mbar, 75° C., 3 h). The reaction vessel is then cooled to 20° C., flushed with nitrogen and charged with dichloromethane (500 mL) and triethylamine (82 g, 810 mmol). The clear solution is then cooled with ice-water to 5° C. and cyclohex-3-ene-1-carbonyl chloride (100.0 g, 691.5 mmol) is added over 50 min while maintaining internal temperature at 5-10° C. (the reaction is exothermic). The turbid reaction mixture is then allowed to warm to 20° C. over 2 h and stirred at this temperature for a further 18 h.

The reaction mixture is then charged into a solution of sodium hydrogen carbonate (50 g) in water (1000 mL) and stirred rapidly for 1 h at 20° C.

The organic phase is separated and extracted with water (350 mL), then again with a solution of sodium hydrogen carbonate (25 g) in water (500 mL) and finally with water (400 mL).

The organic phase is collected and all volatiles are removed in vacuo. The liquid product is then dried to constant weight in vacuo (30 mbar, 50° C.). This provides the desired tetraalkene product (178.7 g).

¹H NMR (400 MHz, CDCl₃): 5.68 (m, 8H), 5.08-5.00 (m, 4H), 3.59-3.23 (m, 28H), 2.58-2.49 (m, 4H), 2.25-1.96 (m, 20H), 1.72-1.62 (m, 4H), 1.23-1.10 (m, 24H).

### Synthesis of a Tetraepoxide Derived from Polyol 4360 (E2

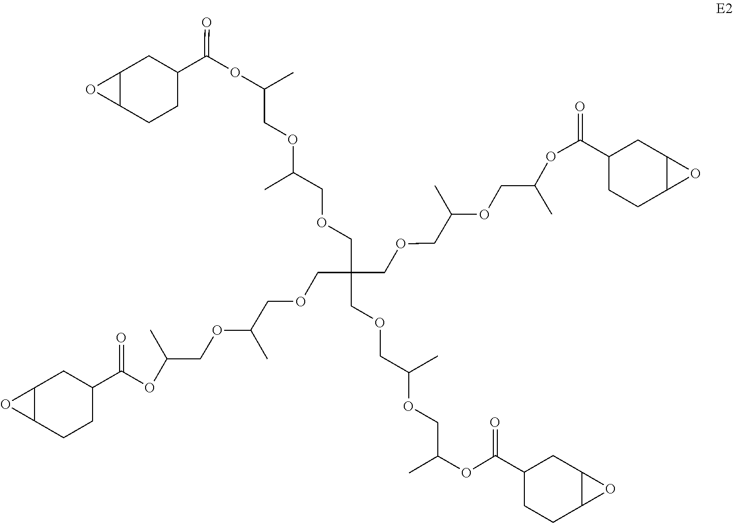 [SMILES: CC(COCC(COCC(C)OCC(C)OC(=O)C1CCC2OC2C1)(COCC(C)OCC(C)OC(=O)C1CCC2OC2C1)COCC(C)OCC(C)OC(=O)C1CCC2OC2C1)OCC(C)OC(=O)C1CCC2OC2C1]

A reaction vessel is charged with the Polyol 4360 tetraalkene (178.7 g, 168.2 mmol) and dichloromethane (1250 mL) is added. The reaction mixture is cooled to an internal temperature of 2° C. 3-chloroperbenzoic acid (72.0% active, 169.3 g, 706.3 mmol) is added over 5 h with vigorous stirring, while maintaining the internal temperature at 3-4° C. The turbid mixture is stirred vigorously for 18 h and allowed to warm to 20° C. The reaction mass is then filtered and the white solid is washed with dichloromethane (2×50 mL). To the obtained filtrate is added a solution of sodium sulphite (50 g) in water (500 mL) and the biphasic mixture is stirred for 60 min. The mixture is phase separated and the organic phase is washed with a solution of sodium hydrogen carbonate (50 g) and sodium sulphite (21 g) in water (500 mL), and then with water (2×500 mL).

The mixture is allowed to phase separate for 18 h and the organic phase is collected, dried with sodium sulphate (220 g) and filtered. The filtrate is concentrated in vacuo and the liquid product is then dried to constant weight (30 mbar, 35° C.). This provides the desired tetraepoxide product E2 (180.6 g, 95.3% of theory).

¹H NMR (400 MHz, CDCl₃): 5.08-4.97 (m, 4H), 3.59-3.10 (m, 36H), 2.54-2.45 (m, 4H), 2.30-1.35 (m, 24H), 1.25-1.06 (m, 24H).

FT-IR (ATR, neat): 2976 (w), 2933 (w), 2871 (w), 1726 (vs), 1376 (m), 1304 (m), 1255 (m), 1231 (m), 1174 (s), 1144 (m), 1100 (vs), 1006 (m), 989 (m), 974 (m), 935 (m), 905 (m), 859 (m), 796 (m), 785 (m).

### Example 3: Synthesis of a Hexaepoxide Derived from Polyol R6405 (E3)

### Synthesis of a Hexaalkene Derived from Polyol R6405

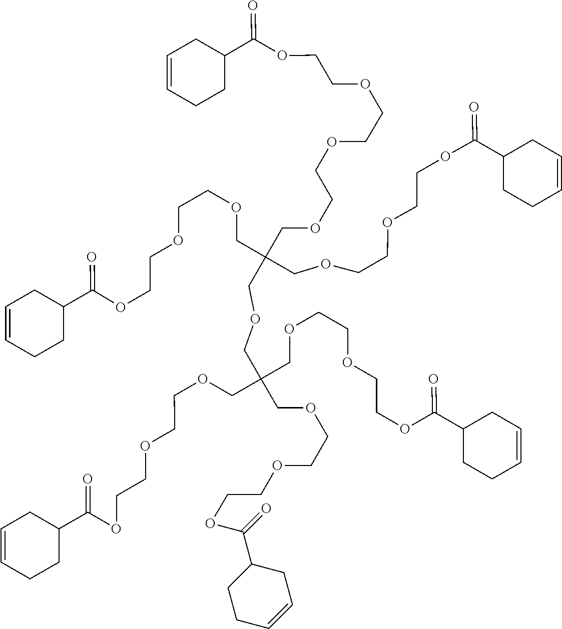 [SMILES: O=C(OCCOCCOCCOCC(COCCOCCOC(=O)C1CC=CCC1)(COCCOCCOC(=O)C1CC=CCC1)COCC(COCCOCCOC(=O)C1CC=CCC1)(COCCOCCOC(=O)C1CC=CCC1)COCCOCCOC(=O)C1CC=CCC1)C1CC=CCC1]

A reaction vessel is charged with Polyol R6405 (385.0 g, 465.5 mmol) and the material is dried under vacuum with stirring (20 mbar, 80° C., 2.5 h). The reaction vessel is then cooled to 20° C., flushed with nitrogen and charged with dichloromethane (2500 mL) and triethylamine (353.3 g, 3.4915 mol). The clear solution is then cooled with ice-water to 12° C. and cyclohex-3-ene-1-carbonyl chloride (445.0 g, 3.077 mol) is added over 2 h while maintaining internal temperature at 12-15° C. (the reaction is exothermic). The turbid reaction mixture is then allowed to warm to 20° C. over 2 h and stirred at this temperature for a further 18 h.

The reaction mixture is then charged into a solution of sodium hydrogen carbonate (205 g) in water (2300 mL) and stirred rapidly for 4 h at 20° C. The organic phase is separated and extracted with water (3×1500 mL).

The organic phase is collected and all volatiles are removed in vacuo. The liquid product is then dried to constant weight in vacuo (30 mbar, 50° C.). This provides the desired hexaalkene product (701.6 g).

¹H NMR (400 MHz, CDCl₃): 5.68 (m, 12H), 4.29-4.06 (m, 12H), 3.71-3.35 (m, 56H), 2.63-2.52 (m, 6H), 2.27-2.22 (m, 12H), 2.14-1.96 (m, 18H), 1.74-1.62 (m, 6H).

FT-IR (ATR, neat): 3025 (w), 2869 (m), 1729 (vs),1303 (m), 1288 (m), 1247 (m), 1222 (s), 1166 (s), 1099 (vs).1064 (s), 1039 (s), 952 (m), 919 (m), 878 (m), 650 (s).

### Synthesis of a Hexaalkene Derived from Polyol R6405 by Direct Esterification

Perstorp Polyol R6405 (515.2 g, 622.97 mmol), 3-cyclohexene-1-carboxylic acid (565.8 g, 4.485 mol) and toluene (2500 mL) are combined in a reaction vessel fitted with a condenser and Dean-Stark trap. The reaction vessel is flushed with nitrogen, methanesulfonic acid (4.2 g) is added with stirring and the reaction mixture is heated to reflux (internal temperature 114-116° C.).

Water is removed from the reaction by azeotropic distillation over 16.5 h (any toluene collected is replaced). The reaction mixture is cooled to 20° C. and extracted with a solution of sodium hydrogen carbonate (100 g) in water (1500 mL). Further toluene (1000 mL) and water (400 mL) are added and the mixture is allowed to phase separate. The organic phase is separated and washed with 1% aqueous sodium hydrogen carbonate (2×1000 mL), then with water (2×750 mL).

The organic phase is collected and all volatiles are removed in vacuo.

The liquid product is then dried to constant weight in vacuo (12 mbar, 55° C.). This provides the desired hexaalkene product (933.5 g).

### Synthesis of a hexaepoxide Derived from Polyol R6405 (E3

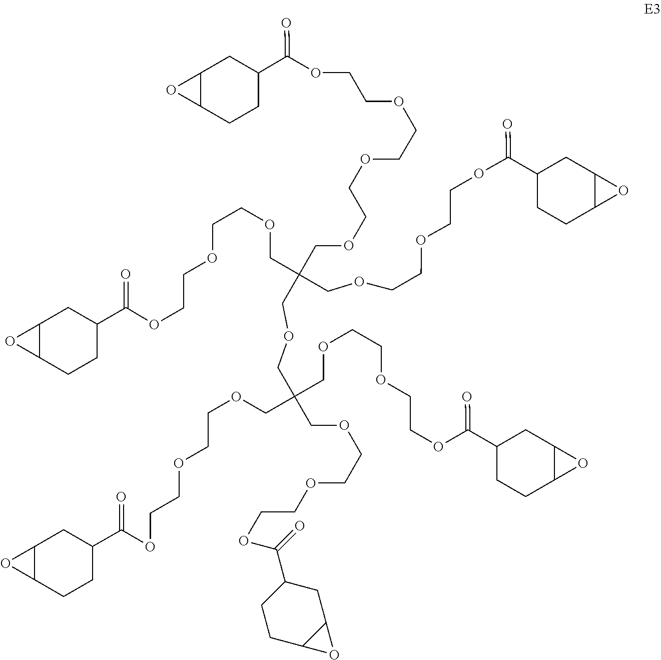 [SMILES: O=C(OCCOCCOCCOCC(COCCOCCOC(=O)C1CCC2OC2C1)(COCCOCCOC(=O)C1CCC2OC2C1)COCC(COCCOCCOC(=O)C1CCC2OC2C1)(COCCOCCOC(=O)C1CCC2OC2C1)COCCOCCOC(=O)C1CCC2OC2C1)C1CCC2OC2C1]

A reaction vessel is charged with the Polyol R6405 hexaalkene (500.0 g, 338.75 mmol) and dichloromethane (2500 mL) is added. The reaction mixture is cooled to an internal temperature of 6° C. and 3-chloroperbenzoic acid (70.2% active, 532.96 g, 2.168 mol) is added over 5 h with vigorous stirring, while maintaining the internal temperature at 4-6° C. The turbid mixture is stirred vigorously for 18 hand allowed to warm to 20° C. The reaction mass is then filtered and the white solid is washed with dichloromethane (350 mL). To the obtained filtrate is added a solution of sodium sulphite (100 g) in water (1500 mL) and the biphasic mixture is stirred for 30 min. The mixture is phase separated and the organic phase is washed with water (2×500 mL). The organic phase is collected and all volatiles are removed in vacuo.

The liquid product is then dried to constant weight in vacuo (30 mbar, 40° C.). This provides the desired hexaepoxide product E3 (492.1 g, 92.5% of theory).

¹H NMR (400 MHz, CDCl₃): 4.21-4.00 (m, 12H), 3.66-3.30 (m, 56H), 3.21-3.09 (m, 12H), 2.54-2.43 (m, 3H), 2.27-1.31 (m, 39H).

FT-IR (ATR, neat): 2868 (m), 1727 (vs),1305 (m), 1255 (m), 1231 (m), 1215 (m), 1173 (s), 1100 (vs),1058 (s), 1015 (m), 991 (m), 974 (m), 936 (m), 904 (m), 874 (m), 859 (m), 837 (m), 796 (m), 786 (m).

### Comparative Example 1

For comparison, the commercially available epoxy resin UviCure S105E and oxetane UviCure 5130 (available from Sartomer) were used.

The difunctional cycloaliphatic epoxide, sold as UviCure® S105 has the following structure:

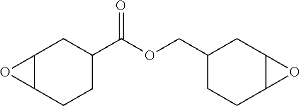 [SMILES: O=C(OCC1CCC2OC2C1)C1CCC2OC2C1]

### Example 4: Curing Experiments

The following example illustrates the UV curing speed of the epoxy resins of the present invention. For all curing tests below, photoinitiator SpeedCure 938 (Sartomer) was used.

Formulations were cured at 100 μm film thicknesses under Hg lamp using a belt curing instrument (Jenton International Ltd., model #JA2000VPXI-0000) with belt speed 15 m/min and 50% lamp intensity (UV dose for 1 pass: UW: 58 mJ/cm², UVA: 108 mJ/cm², UVB: 108 mJ/cm², UVC: 20 mJ/cm²). The substrate used was standard black and white paper (Leneta form 3N-31). Viscosity measurements were performed on a Brookfield viscometer (spindle No. 31, 25° C.). Each tested epoxy resin E1, E2 or E3 was mixed with UviCure 5130 at different weight ratios from 0-100 wt %. The data obtained for mixtures of UviCure S105E and UviCure 5130 were used as control. Photoinitiator SpeedCure 938 (Sartomer) was used at a loading of 1 wt % in the tested resin mixtures.

The cure speed was assessed by the number of passes under the lamp required to give a surface cured ‘tack-free’(TF) coating (as determined when the surface of the coating no longer feels sticky when lightly touched) or depth cure as determined by the ‘thumb-twist’ test (TT) (i.e. until no visible mark is made when a thumb is pressed down firmly onto the coating with a twisting motion). Solvent resistance was tested on the cured samples using MEK double rub test as per ASTM D4752. The results are given in Tables 1, 2, 3 and 4.

TABLE 1

| Example 1: | UviCure | | | | MEK |
|---|---|---|---|---|---|
| Triepoxide | S130 | Viscosity | surface cure | depth cure | double |
| E1 (wt %) | (wt %) | (cP) | (# passes) | (# passes) | rubs |
| 100 | 0 | 950 | — | — | — |
| 90 | 10 | 550 | 15 | 13 | 2 |
| 80 | 20 | 375 | 12 | 9 | 5 |
| 70 | 30 | 238 | 9 | 7 | 8 |
| 60 | 40 | 170 | 7 | 6 | 10 |
| 50 | 50 | 115 | 15 | 5 | 8 |
| 40 | 60 | 82 | No cure | 7 | — |
| 30 | 70 | 60 | No cure | No cure | — |
| 20 | 80 | 43 | — | — | — |
| 0 | 100 | 25 | — | — | — |

TABLE 2

| Example 2: | UviCure | | | | MEK |
|---|---|---|---|---|---|
| Tetraepoxide | S130 | Viscosity | surface cure | depth cure | double |
| E2 (wt %) | (wt %) | (cP) | (# passes) | (# passes) | rubs |
| 100 | 0 | 4900 | — | — | — |
| 90 | 10 | 1700 | 3 | 5 | 8 |
| 80 | 20 | 800 | 2 | 3 | 40 |
| 70 | 30 | 412 | 1 | 3 | 100 |
| 60 | 40 | 230 | 1 | 3 | 50 |
| 50 | 50 | 140 | 2 | 3 | 45 |
| 40 | 60 | 90 | 2 | 3 | 10 |
| 30 | 70 | 60 | 3 | 6 | 10 |
| 20 | 80 | 43 | No cure | No cure | — |
| 0 | 100 | 25 | — | — | — |

TABLE 3

| Example 3: | UviCure | | | | MEK |
|---|---|---|---|---|---|
| Hexapoxide | S130 | Viscosity | surface cure | depth cure | double |
| E3 (wt %) | (wt %) | (cP) | (# passes) | (# passes) | rubs |
| 100 | 0 | 3600 | — | — | — |
| 90 | 10 | 1475 | 5 | 6 | 50 |
| 80 | 20 | 750 | 3 | 4 | >100 |
| 70 | 30 | 400 | 2 | 3 | >100 |
| 60 | 40 | 258 | 2 | 3 | 70 |
| 50 | 50 | 187 | 1 | 2 | 60 |
| 40 | 60 | 95 | 2 | 3 | 32 |
| 30 | 70 | 65 | 3 | 5 | 8 |
| 20 | 80 | 45 | No cure | No cure | — |
| 0 | 100 | 25 | — | — | — |

TABLE 4

| Comparative | | | | | |
|---|---|---|---|---|---|
| Example 1: | UviCure | | surface | depth | MEK |
| UviCure | S130 | Viscosity | cure | cure | double |
| S105E (wt %) | (wt %) | (cP) | (# passes) | (# passes) | rubs |
| 100 | 0 | 250 | 3 | 6 | 2 |
| 90 | 10 | 160 | 2 | 3 | 1 |
| 80 | 20 | 110 | 1 | 2 | 3 |
| 70 | 30 | 80 | 1 | 2 | 5 |
| 60 | 40 | 65 | 1 | 2 | 8 |
| 50 | 50 | 50 | 1 | 2 | 6 |
| 40 | 60 | 45 | 1 | 2 | 7 |
| 30 | 70 | 40 | 2 | 3 | 6 |
| 20 | 80 | 35 | No cure | No cure | — |
| 0 | 100 | 25 | — | — | — |

### Example 5: Polymerization of Alkoxylated Cycloaliphatic Epoxides in Presence of (Meth)Acrylates

### Materials

The list of materials used in the example is detailed in the following Table.

TABLE 5

| Material | Composition | Supplier |
|---|---|---|
| SR833 | 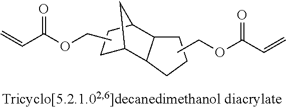 | Sartomer |
| Uvicure S105 (ECC) | 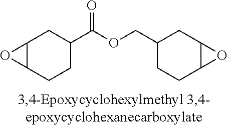 [SMILES: O=C(OCC1CCC2OC2C1)C1CCC2OC2C1] | Sartomer |
| Uvicure S130 (OXT) | 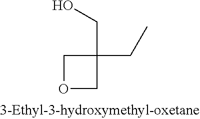 | Sartomer |
| Speedcure BKL | 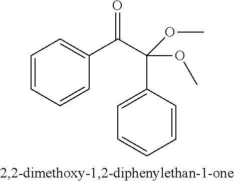 [SMILES: COC(OC)(C(=O)c1ccccc1)c1ccccc1] | Sartomer |
| Speedcure 938 | 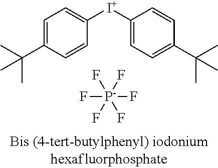 [SMILES: CC(C)(C)c1ccc([I+]c2ccc(C(C)(C)C)cc2)cc1 ; F[P-](F)(F)(F)(F)F] | Sartomer |
| Speedcure ITX | 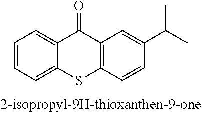 [SMILES: CC(C)c1ccc2sc3ccccc3c(=O)c2c1] | Sartomer |

### Formulations and Results

Formulations were prepared with the ingredients listed in the following tables (amounts are indicated in parts by weight).

TABLE 6A
Summary of formulations with and their properties

| | Comparative | | |
|---|---|---|---|
| Component | Example 2A | Example 4 | Example 5 |
| SR833 | 65.00 | 65.00 | 65.00 |
| UviCure S130 | 5.76 | 5.76 | 5.76 |
| UviCure S105 | 23.04 | | 11.52 |
| Hexaepoxide E3 according to | | 23.04 | 11.52 |
| example 3 | | | |
| SpeedCure BKL | 2.00 | 2.00 | 2.00 |
| SpeedCure 938 | 4.00 | 4.00 | 4.00 |
| SpeedCure ITX | 0.20 | 0.20 | 0.20 |
| Total | 100.00 | 100.00 | 100.00 |
| Viscosity (@ 25° C.), cPs | 102.5 | 220 | 180 |
| Tensile Strength (MPa) | 41.81 | 34.82 | 44.85 |
| Elongation at Break (%) | 1.83 | 3.36 | 2.58 |
| Young's Modulus (MPa) | 2721 | 1717 | 2306 |
| Maximum Load (N) | 328.3 | 247 | 341.2 |
| Energy at Break (J) | 0.177 | 0.272 | 0.268 |
| Acrylate Conversion (%) after | 81.18 | 85.36 | 85.3 |
| 7-days of aging | | | |
| Epoxide Conversion (%) after | 83.41 | 84.7 | 83.48 |
| 7-days of aging | | | |
| 25° C. Storage Modulus, MPa * | 3134 | 2906 | 3119 |
| Loss Modulus peak, ° C. * | 80.59 | 39.88 | 59.77 |
| Tan delta peak, ° C. * | 110.45 | 65.61 | 81.75 |
| Max heat flow (W/g) (365 nm) | 52.68 | 44.72 | 47.44 |
| Peak time (mins) (365 nm) | 0.02 | 0.02 | 0.02 |
| Volume shrinkage (%) | 2.95 | 1.66 | — |

* Post-cured thin tensile and DMA strips 2 × 15 min at 30° C. with 100% intensity in EnvisionTEC PCA 2000 curing unit.

TABLE 6B
Summary of formulations with and their properties

| | Comparative | | |
|---|---|---|---|
| Component | Example 2B | Example 6 | Example 7 |
| SR833 | 65.00 | 65.00 | 65.00 |
| UviCure S130 | 5.76 | 5.76 | 5.76 |
| UviCure S105 | 23.04 | 11.52 | |
| Hexaepoxide E1 according to | | 11.52 | 23.04 |
| example 1 | | | |
| SpeedCure BKL | 2.00 | 2.00 | 2.00 |
| SpeedCure 938 | 4.00 | 4.00 | 4.00 |
| SpeedCure ITX | 0.20 | 0.20 | 0.20 |
| Total | 100.00 | 100.00 | 100.00 |
| Viscosity (@ 25° C.), cPs | 102.5 | 150.0 | 195.0 |
| Tensile Strength (MPa) ^{a} | 32.28 | 35.22 | 37.14 |
| Elongation at Break (%) ^{a} | 1.99 | 3.41 | 3.73 |
| Young's Modulus (MPa) ** | 1996.83 | 1382.46 | 1467.93 |
| Maximum Load (N) ** | 224.9 | 282.46 | 263.9 |
| Energy at Break (J) ** | 0.133 | 0.263 | 0.297 |
| Acrylate Conversion (%) after | 65.96 | 63.70 | 65.56 |
| 7-days of aging | | | |
| Epoxide Conversion (%) after | 84.93 | 83.13 | 87.73 |
| 7-days of aging | | | |
| Tan delta peak, ° C. ^{a} | 98.8 | 88.8 | 79.3 |

** Post-cured thin tensile and DMA strips 2 × 30 min at 60° C. with 100% intensity in Sprintray ProCure curing unit

### Preparation of Formulation

Formulations of Tables 6A and 6B were prepared as follows:

In a 125 mL brown amber glass bottles, the epoxides and oxetanes were loaded first, followed by the photoinitiators. The 100 g mixture of each sample was sealed in bottle by securing lid with white tape. Then, three glass bottles were placed on roller in 65° C. oven for about 2 h until the solution became clear.

### Viscosity Measurement

Viscosity measurements were taken using a Brookfield DV-II+ Pro viscometer. A standard S18 size spindle was used to measure viscosity. The viscosity readings were taken at 25° C., with the torque % between 30-80%.

### Printing Thin Strip Sample Via 355 nm SLA Viper

a. Cut the PET film (approximate size of 7.5″×6.5″) into a square which fits the shape of the glass (8″×8″) and attached the PET film to the glass using the double-sided tape.
b. Sprayed the non-sticky substances (Rust-o-leum Never Wet Coat 1 Spray) onto the PET film evenly.
c. Selected the ‘elevator motion’ of SLA Viper to set the elevator position as 2.1740.
d. Used the pipette to draw around 2 mL liquid onto the glass.
e. Used 5 μm side of coating applicator to apply the first layer of film.
f. Set up a moderate E_{c} and Dₚ (E_{c}=65, Dₚ=5 for all three formulations), start printing
g. Once the first layer of printing completed, used the 10 μm side of applicator to apply the second 5 μm of layer of film.
h. Repeated printing and applying thin film until 30 μm side had been used.
i. Thus, a 6 layer, 5 μm each layer, three 4″×0.5″ green thin strips and one 35 mm×12 mm DMA strip were printed per print.
j. Drain off uncured liquid resin, clean with IPA and dry in air
k. Carefully peel each thin strip from PET film
l. Post-cure thin strips according to the conditions detailed under Tables 6A and 6B
m. Stored at 23±2° C. and 50±10% relative humidity conditions for at least 7 days before test

### Tensile Test

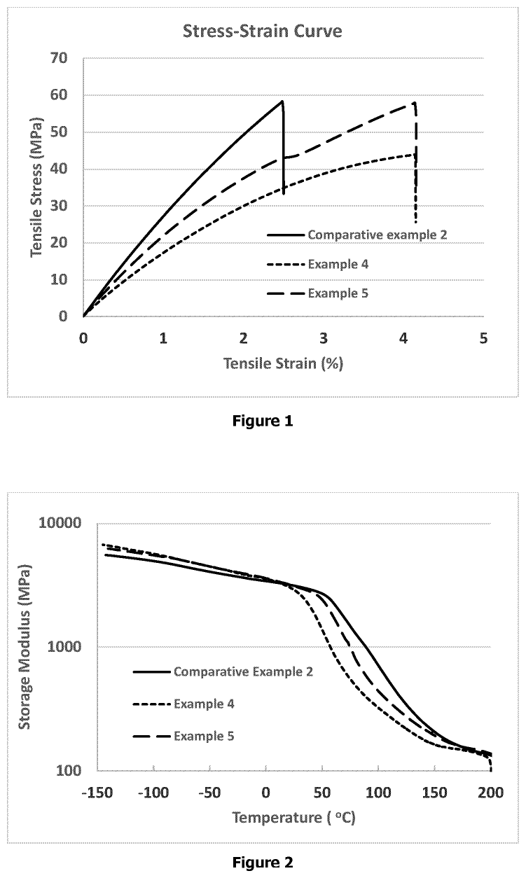

An Instron 5966 with load capacity of 10 kN with tensile testing fixtures was used to measure tensile properties. The thin strips are prepared for tensile properties. Six specimens were prepared for each sample, and the test speeds for tensile tests were set to 5 mm/min. Tests were conducted following the ASTM D882 protocol. Results are shown in Tables 6A and 6B and FIG. 1.

### DMA Test

Using a TA Instruments DMA Q800, changes were observed in mechanical properties in each cured DMA strip throughout a temperature range. To study materials in this scope a program in DMA that operates from −150° C. to 250° C. at 3° C./min with a frequency of 1 Hz is used. Resulting storage modulus (G′), loss modulus (G″) and tan(delta) curves were analyzed to understand changes in polymer behavior. Results are shown in Tables 6A and 6B and FIG. 2.

### FTIR Testing

A Fourier Transform Infrared (FTIR) with an Attenuated Total Reflection (ATR) setup was used. All polymerization rate measurements were performed using Nicolet iS50 FT-IR Spectrometer from Thermo Scientific, equipped with a standard DLaTGS detector. For measurement, a drop of liquid sample was placed in the center of an ATR crystal to collect IR spectrum, then a flat surface of a printed and cured thin strip was pressed over ATR crystal to collect a new IR spectrum for both acrylate and epoxy conversion calculation. Measurements were taken at the area under the reference peak around 1720 cm⁻¹; the acrylate peak at approximately 1407 cm⁻¹ and the epoxy peak at approximately 790 cm⁻¹ were also measured. Peak area was determined using the baseline technique where a baseline is chosen to be tangent to absorbance minima on either side of the peak. The area under the peak and above the baseline was then determined. The integration limits for liquid and the cured sample are not identical but are similar, especially for the reference peak. The ratio of the acrylate or epoxy peak area to the reference peak were determined for both the liquid and the cured samples. Degree of cure or conversion, expressed as percentage reacted acrylate or epoxy, was calculated from the equation below:

$$Conversion ( \% ) = [ ( {R}_{l i {q}^{-}} {R}_{c} ) \times 100 ] / {R}_{liq}$$

Where R_{liq} is the area ratio of the liquid sample and Re is the area ratio of the cured tensile strip. The resulting acrylate and epoxy conversions were tested using the FTIR method described as above. The results are shown Tables 6A and 6B.

### LED-DSC Test

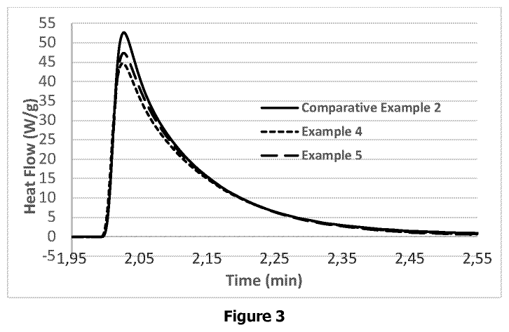

A photo differential scanning calorimetry (DSC) with an customized 365 nm LED lamp setup was used. All photopolymerization rate measurement were performed using a Q2000 DSC unit from TA Instruments. A lamp holder for the DSC unit can be customized and printed from Arkema N3xtDimention® engineered resin N3D-TOUGH784 in order to ensure precisely fit of a 365 nm lamp Accucure ULM-2-365 from Digital Light Labs. The LED light was automatically triggered by connecting the “Event” outlet of the DSC unit to an Accure Photo Rheometer Ultraviolet Illumination & Measurement System. LED light exposure can be programed by using “Event” on or off from Photo DSC software, but the intensity of light can be preset from the Accure Photo Rheometer Ultraviolet Illumination & Measurement System. For measurement, approximate 5 mg liquid sample was placed at the center of a T130522 DSC Tzero pan, cured by exposing it to 50 mW/cm² of 365 nm LED light for 5 minutes under a 50 mL/min N2 flow rate and 45° C. The resulting heat flow (W/g) curve was collected to analyze maximum heat flow peak value and maximum peak time. Results are shown in Tables 6A and 6B and FIG. 3.

### Volume Shrinkage Test

A. Determination of density for formulated resins:
   a. After formulating and mixing of a resin, fill a pre-weighed 5 mL or 10 mL volume flask with such resin to the mark.
   b. Weigh the whole volume flask and calculate the density by dividing the net weight of the formulated rein by the volume.
   c. Repeat for 3 times of this step and take the mean value as the density of the formulated resin (as D, unit: kg/L).
B. Shrinkage measurement method is based on volumetric principle:
   a. Certain weight of formulated resin (as W1) was carefully dispersed into pre-weight aluminium foil weighing boat to fill the boat to the height about 3-5 mm.
      Make sure there is bubble trapped inside the resin.
      The volume of the foil weighing boat is limited to about 1 mL each. This is to make sure the cured resin piece is narrow enough to pass through the neck of a volume flask.
   b. The curing was conducted under mercury lamp condition for enough passes to get surface cured.
   c. The foil boat was then put into 60° C. oven for further depth curing until full cure.
   d. Take the foil boats out of oven and wait until they cool to room temperature.
   e. Fill a 50 mL or 100 mL volume flask with DI-water to the mark.
      Peel off the foil and take the cured resin into the volume flask.
      The resin should be fully immersed and sink. Shake to make sure no air bubble stays inside the flask.
   f. Take the volume flask on analytical balance, tare zero. Use a pipette to suck out excess water and make the water volume to the mark again. Record the weight loss for the whole volume flask (as W2).
   g. At least three replicates are needed for each of the resin formulation.
      Then calculation was done to get the weight loss of the volume flask into water volume. The calculated water volume should be equal to the final volume of cured resin according to the test design. The water density applied is 0.998 kg/L @20° C.
C. Volume shrinkage ratio is calculated by following equation:

$$Shrinkage \% = ( ( W 1 / D ) - ( W 2 / 0.998 ) ) / ( W 1 / D ) \times 100 \%$$

### Test Results and Discussion

Comparing with 3,4-Epoxycyclohexylmethyl-3,4-epoxycyclohexanecarboxylate (ECC) in a hybrid system, Table 6A results showed hexa-epoxide according to the invention slightly increased viscosity of formulation, improved tensile toughness as showed in FIG. 1, lowered down glass transition temperature from DMA test in FIG. 2, and slow down cure speed as characterized by heat flow from LED-DSC test in FIG. 3.

Comparing with 3,4Epoxycyclohexylmethyl-3,4-epoxycyclohexanecarboxylate (ECC) in a hybrid system, Table 68 results showed tri-epoxide according to the invention also slightly increased viscosity of formulation, improved tensile toughness, lowered down glass transition temperature as characterized by DMA test.

1. An alkoxylated cycloaliphatic epoxide according to the following formula (I): 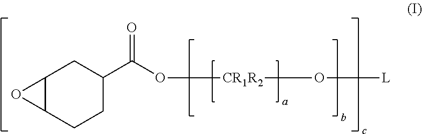
wherein
each R₁ and R₂ is Independently selected from H and Me;
L is the residue of a polyol;
each a is independently from 2 to 4;
each b is independently 0 to 20 with the proviso that at least one b Is not 0;
c is at least 3.

2. The alkoxylated cycloaliphatic epoxide according to claim 1, wherein a is 2 and the alkoxylated cycloaliphatic epoxide is according to the following formula (Ia): 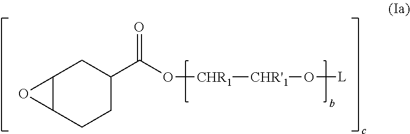
wherein
L, b and c are as defined in claim 1;
each R₁ and R′₁ is independently selected from H and Me.

3. The alkoxylated cycloaliphatic epoxide according to claim 1, wherein a is 4 and R₁ and R₂ are both H.

4. The alkoxylated cycloaliphatic epoxide according to claim 1, wherein each b is independently from 1 to 20.

5. The alkoxylated cycloaliphatic epoxide according to claim 1, wherein the alkoxylated cycloaliphatic epoxide has an alkoxyation degree of at least 6.

6. The alkoxylated cycloaliphatic epoxide according to claim 1, wherein c is from 3 to 10.

7. The alkoxylated cycloaliphatic epoxide according to claim 1, wherein c is 3 and L is a trivalent linker according to the following formula (II): 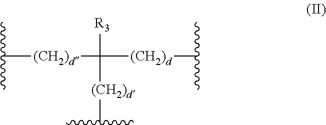
wherein
R₃ is selected from H, alkyl and alkoxy;
d, d′ and d″ are independently 0 to 2 with the proviso that at least 2 among d, d′ and d″ are not 0.

8. The alkoxylated cycloaliphatic epoxide according to claim 1, wherein c is 4 and L Is a tetravalent linker according to one of the following formulae (IIIa), (IIIb) or (IIIc): 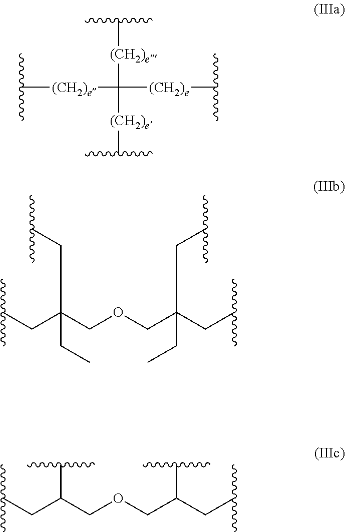
wherein
e, e′, e″ and e′″ are Independently 0 to 2 with the proviso that at least 3 among e, e′, e″ and e′″ are not 0.

9. The alkoxylated cycloaliphatic epoxide according to claim 1, wherein c is 5 and L is a pentavalent linker according to the following formula (IV):

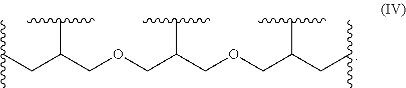 [SMILES: *CC(*)COCC(*)COCC(*)C*]

10. The alkoxylated cycloaliphatic epoxide according to claim 1, wherein c is 6 and L is a hexavalent inker according to the following formula (Va), (Vb) or (Vc):

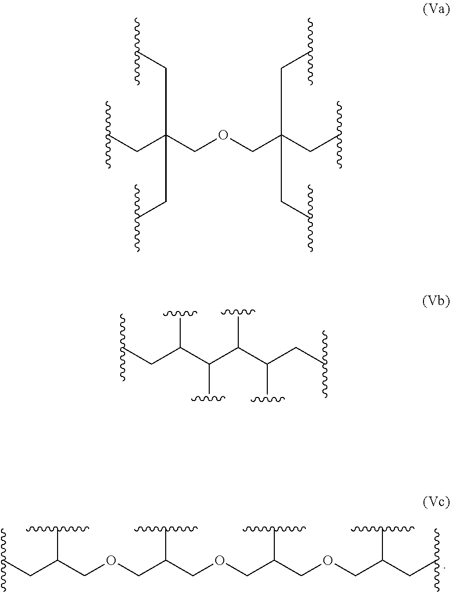

11. A process for the preparation of an alkoxylated cycloaliphatic epoxide of formula (I) as defined in claim 1, wherein the process comprises the following steps:
a) reacting a cyclohexene of formula (VI) with an alkoxylated polyol of formula (VII) to obtain an alkoxylated cyclohexene of formula (VIII);
b) epoxidation of the alkoxylated cyclohexene of formula (VIII) to obtain an alkoxylated cycloaliphatic epoxide of formula (I); 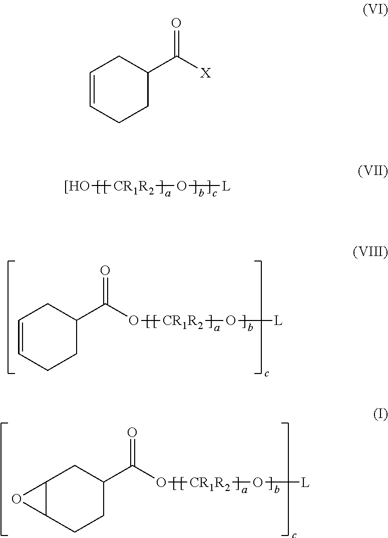
wherein
L, R₁, R₂, a, b and c are as defined in any one of claims 1 to 10;
X is OH, O-Alk or Cl;
Alk Is a C1-C6 alkyl.

12. A composition comprising a mixture of alkoxylated cycloaliphatic epoxides of formula (I): 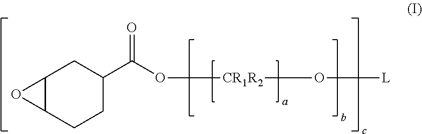
wherein
L, R₁, R₂, a and b are as defined in claim 1;
c is at least 2; and
at least one of the alkoxylated cycloaliphatic epoxides of formula (I) in the mixture has c is at least 3.

13. A composition comprising:
a) at least one alkoxylated cycloaliphatic epoxide of formula (I) according to claim 1; and
b) at least one cationically polymerizable compound other than component a).

14. The composition of claim 13, wherein component b) comprises at least one cationically polymerizable compound selected from the group consisting of an epoxy-functionalized compound other than component a), an oxetane, an oxolane, a cyclic acetal, a cyclic lactone, a thiirane, a thietane, a spiro orthoester, a spiro orthocarbonate, a vinyl ether, a vinyl ester, derivatives thereof and mixtures thereof.

15. The composition of claim 13, wherein component b) comprises at least one oxetane according to the following formula (IX): 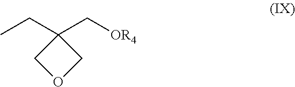
wherein R₄ is selected from H, alkyl, aryl, alkylaryl, (meth)acryloyl, —CH₂—CH₃, —L₁-O—CH₂-oxetanyl —CH₂—CH₃,
L₁ is a divalent linker, in particular-CH₂-Ph-Ph-CH₂ or —CH₂-Ph-CH—[O—CH₂-Ph-CH₂]_{f}—
Ph is phenylene;
f is 0 to 10.

16. The composition according to claim 15, wherein component b) comprises at least one oxetane according to formula (IX) wherein R₄ is H, benzyl or —CH₂-oxetanyl-CH₂—CH₃.

17. The composition according to claim 13, wherein the weight ratio between component a) and component b) is 20:80 to 80:20.

18. The composition according to claim 13, wherein the composition comprises at least one cationic photoinitiator.

19. A composition comprising:
a) at least one alkoxylated cycloaliphatic epoxide of formula (I) according to claim 1; and
c) at least one (meth)acrylate-functionalized compound.

20. The composition according to claim 19, wherein the composition comprises at least one free-radical photoinitiator.

21. The composition according to claim 13, wherein the composition is an ink, coating, sealant, adhesive, molding, or 3D printing composition.

22. A process for the preparation of a cured product, comprising curing the composition according to claim 19, by exposing the composition to radiation such as UV, near-UV, visible, Infrared and/or near-infrared radiation or to an electron beam.

23. (canceled)

24. A cured product obtained by curing the composition according to claim.

25. A cured product according to claim 24, wherein the cured product is an ink, a coating, a sealant, an adhesive, a molded article or a 3D-printed article.

26. (canceled)
